(19) United States
(12) Patent Application Publication
(10) Pub. No.: US 2025/0284910 A1
(43) Pub. Date: Sep. 11, 2025

(54) SUPER-RESOLVED OBJECT DETECTION WITH SPATIAL GENOMICS

(71) Applicant: CALIFORNIA INSTITUTE OF TECHNOLOGY, Pasadena, CA (US)

(72) Inventors: Jonathan A. WHITE, Pasadena, CA (US); Long CAI, Pasadena, CA (US)

(21) Appl. No.: 19/052,266

(22) Filed: Feb. 12, 2025

Related U.S. Application Data
(60) Provisional application No. 63/552,608, filed on Feb. 12, 2024.

(51) Int. Cl. G06K 7/14; G16B 40/10

(52) U.S. Cl. CPC G06K 7/1443; G06K 7/1413; G16B 40/10

(57) ABSTRACT

The present disclosure provides methods for encoding and decoding signals from barcodes in a plurality of molecular targets from images obtained from imaging based spatial genomics (ISG) experiments. This disclosure sets forth methods, in addition to use of the same, and other solutions to problems in the relevant field.

### CROSS-REFERENCE TO RELATED APPLICATION

The present application claims the benefit of U.S. Provisional Application No. 63/552,608, filed Feb. 12, 2024, and incorporates the entirety of that application by reference.

### STATEMENT REGARDING FEDERALLY-SPONSORED RESEARCH

This invention was made with government support under Grant No(s). R01AG066028A awarded by the National Institutes of Health. The government has certain rights in the invention.

### BACKGROUND

Spatial genomics technologies can measure gene expression of cells in their native tissue context, promising to shed light on interactions between cells. Sequential fluorescent in-situ hybridization (seqFISH) (Lubeck et al. April 2014) is a spatial genomics technology that builds on single-molecule FISH (smFISH) (Femino et al. 1998; Raj et al. October 2008). In imaging based spatial genomics experiments, many cycles of probe hybridization, imaging, and probe stripping are performed. In some implementations, RNAs from each gene are probed in one hybridization cycle, providing the pseudo-color of each barcoding block. Unique combinations of pseudo-colors represent a codeword for each gene (Eng et al. December 2017; Shah et al. July 2018; Eng et al. April 2019).

Traditionally, FISH based spatial transcriptomics data has been processed using either spot-based decoding (Femino et al. 1998; Raj et al. October 2008; Lubeck et al. April 2014; K. H. Chen et al. 2015; Shah et al. July 2018; Eng et al. April 2019; Gataric et al. 2021) or pixel-based decoding (Moffitt et al. 2016; S. Chen et al. 2021; Andersson et al. 2021). The spot-based method used in (Eng et al. December 2017), (Shah et al. July 2018), and (Eng et al. April 2019) can handle dense imaging based spatial genomics datasets that include tens of thousands of genes.

Resolving spatially overlapping barcodes is a major factor in limiting the throughput and accuracy of imaging-based spatial genomics (ISG) methods. This problem arises when either a large number of genes are multiplexed or highly expressed genes are probed in an experiment. Previous approaches to resolve this density problem have used probabilistic or deterministic super-resolution methods, as well as expansion microscopy. However, these methods are unable to resolve ambiguities that arise when multiple barcodes overlap spatially.

### SUMMARY

The present disclosure provides methods for encoding and decoding signals from barcodes in a plurality of molecular targets from images obtained from ISG experiments. This disclosure sets forth methods, in addition to use of the same, and other solutions to problems in the relevant field. The present disclosure provides methods to disentangle overlapping barcodes using global optimization approaches.

In some embodiments a method is described comprising a computer-implemented method to assign barcodes from images of dots representing fluorescent probes interacting with molecular targets, to a plurality of molecular targets. In some embodiments, the method comprises, using a computer, tracing dots between images within a search radius (r) and assigning dots to barcodes to form a plurality of candidate barcodes. In some embodiments, the method comprises, using a computer, assigning a cost to the plurality of candidate barcodes to obtain an assigned cost. In some embodiments, the method comprises, using a computer, for the plurality of candidate barcodes, assigning a penalty to unused dots in each of the images to obtain a penalty cost. In some embodiments, the method comprises, using a computer, choosing a trial solution, the trial solution comprising a set of candidate barcodes with the smallest cost. In some embodiments, the method comprises, using a computer, assigning the candidate barcodes, selected by the trial solution, with the smallest cost to the molecular targets.

Though both spatial transcriptomics and telecommunications both use error-correcting codes, the technologies face different challenges. The goal of telecommunication is to robustly encode long messages (>>10 symbols) when all messages are transmitted one at a time in a channel, and corruptions occur while in transit. In contrast, spatial transcriptomics only needs to encode short messages (˜10 symbols encoding a subset of 20,000 genes), but the barcodes can overlap spatially, giving rise to ambiguity. Spatial transcriptomics can be analogized to telecommunications in that multiple messages are being transmitted in the same channel at the same time.

Difficulty in resolving spatially overlapping barcodes is a major factor limiting the throughput and accuracy of ISG methods. The methods described herein provide an approach that uses strong encoding to reduce the number of spurious solutions that arise from superimposed barcodes. The methods described herein then resolve ambiguities through global optimization. The methods demonstrate an improved performance in simulation and experiment.

The examples provided herein demonstrate an approach for processing ISG data that substantially improves results and promises to enable encoding and decoding barcodes. After registering images, preprocessing the images, and identifying the dots in them, the next step for processing ISG data is to infer which barcoded objects (barcodes) generated observed dots after they have been aligned. Symmetric nearest neighbors decoding can suffer when used on dense datasets because it has combinatorial complexity and because it may have difficulty discriminating spurious solutions that arise when dots read out from multiple barcodes align with each other or with dots generated from non-specifically bound probes. The embodiments described herein provide methods to resolve such ambiguous cases that arise when two or more barcoded target objects colocalize.

The examples provided herein demonstrate how to design ISG barcodes using Reed-Solomon codes, a class of error correcting codes that are Maximum Distance Separable. This means that their codewords differ from one another by the maximum amount possible for the number of redundant parity check symbols that they contain. This reduces the frequency and increases the target object density at which ambiguities arise. The methods provide a way to resolve ambiguous situations that may arise by first identifying all candidate barcodes that could reasonably have been generated by the observed dots, and then finding the globally optimal set of barcodes that explains the observations.

The methods provided herein demonstrate that encoding with Reed-Solomon codes and decoding with global optimization can resolve ambiguity from overlaps allowing “super-resolution in coding space” under conditions where barcode completion is high, which holds for almost all spatial transcriptomics methods. This disclosure further clarifies the differences in the coding schemes used in the literature with an advantage of the barcoding block-based coding schemes compared to the binary or non-linear coding schemes.

### BRIEF DESCRIPTIONS OF THE DRAWINGS

FIG. 1. (A) Candidate barcodes are identified by summing syndromes from numbered pseudo-colors of dots that align in subsequent barcoding blocks. Dots in the first barcoding block are assigned arrays holding their pseudo-color numbers. Dots in the second barcoding block are assigned an array found by adding its pseudo-color number element-wise modulo 20 to the concatenated arrays of dots in the first barcoding block that align within a search radius. This procedure is repeated for dots in the third and fourth barcoding blocks, but subtracting their pseudo-color number modulo-20 instead of adding. Weighted sums evaluating to zero in the fourth barcoding block indicate perfect codepaths which can be traced back to find their contributing dots. (B) shows candidate barcodes identified by syndrome decoding in a real seqFISH+ data (Eng, 2019). Images of each barcoding block show pixels pseudo-colored according to the aligned fluorescent signal of each pseudo-colored image. The locations of dots centered in the region of interest are marked by circles of their pseudo-color and labeled with the number of their pseudo-color. Valid barcodes are connected by lines of a color unique to each barcode. The Aldh18a1 barcode (gray) is unambiguous, but the Tagln (blue), Caprin (yellow), and negative control (red) barcodes are conflicting. In this case, it appears that a non-specific dot, pseudo colored 20 in barcoding block 1, introduces a spurious negative control barcode straddling the Tagln and Caprin barcodes. (C) To resolve the conflicts and find the most likely solution, the network of conflicting barcodes was transformed into a graph where each node represents a barcode, and edges represent conflicts. Feasible solutions cannot contain barcodes connected by an edge. Each solution is scored by summing the costs of the barcodes it contains and the costs of not decoding each dot that is not decoded. The lowest cost solution is chosen using integer programming. (D) Different parameters of the decoding objective function tune the efficiency-FDR tradeoff of the procedure.

FIG. 2. (A) Simulations of numerous barcodes in a one-pixel area determine the relative robustness of different error-correcting codes to spurious solutions arising from superimposed barcodes. The dendrogram on the left groups codes by their family first, then various parameters: q, symbol alphabet size; n, number of symbols; md, minimum Hamming distance between any pair of codewords; w, the weight of codewords (i.e. how many dots are in each barcode; h, the number of images required for an experimental implementation; and c, the number of codewords used from the code. (B) 10 superimposed barcodes were correctly decoded in a Reed-Solomon encoded simulation. The correct decoded barcodes are shown in colored paths. The gray paths are the candidate barcodes that are not decoded. In this simulation replicate, the algorithm correctly determined all of the simulated true genes. (C) An illustration of decoding in real data from the cross-channel Reed-Solomon encoded experiment. Z planes are pseudo-colored barcoding block images where each pseudo-color has been registered by an affine transformation. Markers denote the fit locations and pseudo-colors of dots in each barcoding block. Dots connected by black lines represent decoded gene-encoding barcodes. Dots connected by dark red lines represent decoded negative control barcodes. Dots connected by dotted gray lines represent undecoded candidate barcodes. (D) Different parameters of the decoding objective function tunes the efficiency-FDR tradeoff.

FIG. 3. Simulation of probing 542 highly expressed genes encoded with the q7k3w4 Reed-Solomon Code in NIH 3T3 cells where each transcript occurs at the relative frequency that it is found in bulk sequencing data. The simulation area is 10×10 pixels. The position variance penalty is set to 2, and the search radius is calculated using equation 5. Simulating the data in image form reduces both the efficiency and the false discovery rate as image detection algorithms fail to distinguish overlapping dots in the same readout image. The simulation used 30 replicates.

FIG. 4. Provides the numerical performance values plotted in FIG. 3.

FIG. 5. Illustration of the construction of a decoding directed acyclic graph in decoding simulated data encoded with the q7n6k3 Reed Solomon code. One readout from the codeword has been dropped, and a non-specific dot has been added. Directed edges connect aligning dots from dots in higher indexed barcoding blocks to dots in lower indexed barcoding blocks. Edges can span multiple (n−w+t+1) barcoding blocks because barcodes are not probed in every barcoding block, and readouts may fail to hybridize, be detected, or align. Syndromes are summed for paths in the DAG, and intermediate sums for paths of lengths 1 to w passing through each dot are stored. + represents element-wise modulo 7 addition and “⊙” represents element-wise modulo-7 multiplication. Perfect codepaths that perfectly correspond to a codeword, or imperfect codepaths that are error-correctable to a codepath can be identified. In this case, one imperfect codepath is identified, which can be decoded. Thus, an exemplary Reed-Solomon code, with the described decoding algorithm, is able to unambiguously resolve this scenario where a barcode failed readout and a nonspecific dot colocalize.

FIG. 6. (A) An illustration of the simplified dynamic programming algorithm for evaluating syndromes in the traditional q-ary parity check code used in the published 10,000 gene seqFISH+ experiment. Candidate barcodes are identified by summing syndromes from numbered pseudo-colors of dots that align in subsequent barcoding blocks. Dots in the first barcoding block are assigned arrays holding their pseudo-color numbers. Dots in the second barcoding block are assigned an array found by adding its pseudo-color number element-wise to the concatenated arrays of dots in the first barcoding block that align within a search radius. This procedure is repeated for dots in the third and fourth barcoding blocks, but subtracting their pseudo-color number. Sums evaluating to zero in the fourth barcoding block indicate valid barcodes which can be traced back to find their contributing dots. (B) A more general case of the dynamic programming algorithm for syndrome decoding with an error-correcting code. This case differs from the case in panel A in two ways. First, three parity check equations are summed, so three partial sums are stored where a single partial sum would be stored in panel A. Second, zeros symbols are not probed, so the dots are aligned to neighbors in multiple preceding barcoding blocks and the number of dots contributing to each partial sum is tracked. The dots in the second through n−w+1^{st} barcoding block must align with all previous barcoding blocks. Dots in barcoding blocks b∈[n−(w−2), n] must only continue adding to partial sums with neighboring dots containing partial sums of n−b or more dots.

FIG. 7. Expanded version of FIG. 2. The first 8 rows show simulation for codes from the q-ary parity check code family used in previous ISG experiments. The first q-ary parity check code in each coupled pair uses all codewords from the code and the second uses a subset of the codewords, or to match the experimental characteristics of the Reed-Solomon encoded experiment described herein.

FIG. 8. A plot of the number of candidate barcodes arising in simulations of various codes as a function of the number of true barcodes in the simulations. These results are drawn from the same simulations shown in FIG. 7. Codes that produce fewer spurious candidate solutions in the simulations have higher decoding efficiency and lower false discovery rates in the same simulations than codes that produce more spurious candidate solutions.

FIG. 9. Numerical values of the average numbers of simulated barcodes that were correctly decoded using various codes in various simulated conditions shown in FIG. 7.

FIG. 10. Decoding efficiency is measured as the slope of the linear regression fit between average counts of genes found per cell by cross-channel Reed-Solomon encoded seqFISH against the average counts of the same genes found per cell by smFISH on NIH3T3 cells taken from the same culture.

FIG. 11. An empirical cumulative distribution function showing the number of gene encoding barcodes decoded in each cell by cross-channel Reed-Solomon encoded ISG experiment.

FIG. 12. Decoding performance on simulated ISG images and dot locations with various barcode and non-specific dot densities. Barcode and non-specific dot densities are for the entire simulated experiment, not individual images.

FIG. 13. A plot showing the number of true positive and false positive barcodes decoded in simulated images of 25 barcodes with four readouts from the Reed Solomon q11n10k7 code using L0-regulated regression. The simulation was over an area of 5 by 5. The simulation included 20 non-specific dots. Point spread functions were 2D Gaussians with sigma of 1. The location of each barcode was drawn from a uniform distribution on the region of interest. Each readout dot had a 95% chance of being drawn and a 5% chance of being dropped. The y-axis gives the numbers of correctly and incorrectly decoded barcodes as a function of the regularization parameter.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges.

Oligonucleotides can be of various lengths. In particular embodiments, oligonucleotides can range from about 2 to about 1000 nucleotides in length. In some embodiments, the oligonucleotides can range from about 10 to about 100 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In some embodiments, the molecular target comprises a second moiety capable of forming the covalent linkage with the label. In particular embodiments, a label comprises a nucleic acid sequence that is capable of providing identification of the cell which comprises or comprised the molecular target. In some embodiments, a plurality of cells is labelled, wherein each cell of the plurality has a unique label relative to the other labelled cells.

As disclosed herein, the term “barcode” generally refers to a symbol sequence of a label produced by methods described herein. The barcode sequence typically is of a sufficient length and uniqueness to identify a molecular target.

As disclosed herein, the term “pseudo-color” generally refers to the value of symbol from an error-correcting code represented by a dot in an image of a barcoding block.

As disclosed herein, the term “barcoding block” generally refers to a plurality of images from which dots comprising the images represent a symbol of a defined pseudo-colors encoding at the position in the codeword sequence assigned to the barcoding block.

In some embodiments, the term “targets” generally refers to transcripts, RNA, DNA loci, chromosomes, DNA, protein, lipids, glycans, cellular targets, organelles, and any combinations thereof. In some embodiments, the transcripts, RNA, DNA loci, chromosomes, DNA, protein, lipids, glycans, cellular targets, organelles, and any combinations thereof are conjugated to an oligonucleotide.

In some embodiments, the term “dots” generally refers to the signals emitted by the fluorophores on fluorescent probes. In some embodiments, a dot is a signal is emitted by a detectably labelled probe.

As disclosed herein, the term “symbol” refers to part of a codeword.

As disclosed herein, the term “codeword” refers to a sequence of symbols. In certain embodiments, codewords correspond to molecular targets. In certain embodiments, some codewords in the codebook correspond to “empty” or “null” targets, which are not actual molecular targets and can be used as negative controls.

### Overview

The present disclosure provides methods for encoding and decoding signals from barcodes in a plurality of molecular targets from images obtained from spatial genomics experiments. This disclosure sets forth methods, in addition to using the same, and other solutions to problems in the relevant field.

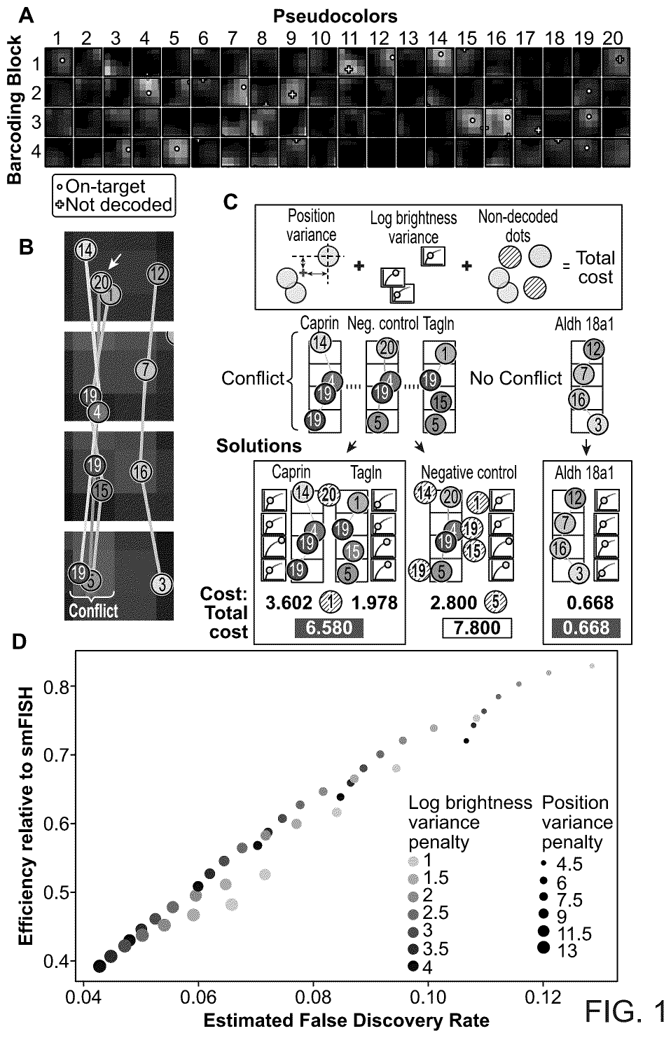

FIG. 1 is representative of some of the embodiments of the methods disclosed.

In FIG. 1, (B), there are four barcoding rounds each consisting of several rounds of fluorescently labelled probes that hybridize to molecular targets. Each barcoding round corresponds to a panel in FIG. 1, (B). In each round, values are assigned to dots. For instance, in the first barcoding round (top panel) values are assigned to each dot (14, 20, 1, and 12). In the next barcoding round, the dots are assigned values of 7, 19, and 4. In the third barcoding round, the dots are assigned values of 19, 15, and 16. In the final barcoding round dots are assigned values of 19, 5, and 3. Dots are traced through each image creating candidate barcodes. In this case, in FIG. 1, (B), there are four candidate barcodes created as shown by the line tracing. Three of the candidate barcodes have conflicts.

FIG. 1, (C) analyzes the candidate barcodes to assign the candidate barcodes with the smallest cost to the molecular targets. Focusing on the three candidate codes with conflicts, in FIG. 1, (C), the Tagln and Caprin have 1 trial solution with two barcode candidates. The third barcode candidate is a negative control with another trial solution. In this example, a cost function is used to resolve the conflict between the two conflicting trial solutions. This is done by assigning a cost to each barcode candidate and then penalizing the candidate for dots that are unused. The negative control is penalized 5 unused dots. Whereas the Caprin and Tagln barcode candidates are penalized one dot. The total cost for the trial solution for the Caprin and Tagln molecular targets is lower (6.58) than the negative control (7.8). The candidate barcodes, selected by the trial solution with the smallest cost, are then assigned to their molecular targets.

In some embodiments a method is described comprising a computer-implemented method to assign barcodes from images of dots representing fluorescent probes interacting with molecular targets, to a plurality of molecular targets. In some embodiments, the method comprises, using a computer, tracing dots between images within a search radius (r) and assigning dots to barcodes to form a plurality of candidate barcodes. In some embodiments, the method comprises, using a computer, assigning a cost to the plurality of candidate barcodes to obtain an assigned cost. In some embodiments, the method comprises, using a computer, for the plurality of candidate barcodes, assigning a penalty to unused dots in each of the images to obtain a penalty cost. In some embodiments, the method comprises, using a computer, choosing a trial solution, the trial solution comprising a set of candidate barcodes with the smallest cost. In some embodiments, the method comprises, using a computer, assigning the candidate barcodes, selected by the trial solution, with the smallest cost to the molecular targets.

In some embodiments, the method of any of the previous embodiments further comprises, for each candidate barcode in the plurality of candidate barcodes, adding the assigned cost to the penalty cost.

In some embodiments, the method of any of the previous embodiments further comprises summing the assigned cost and the penalty cost for each of the plurality of candidate barcodes to obtain a respective total cost.

In some embodiments, the method of any of the previous embodiments, further comprises resolving conflicting candidate barcodes that use at least one of the same dots.

### Tracing Dots Between Images

In some embodiments, the method comprises, using a computer, tracing dots between images within a search radius (r) and assigning dots to barcodes to form a plurality of candidate barcodes.

As shown in FIG. 1, (B), dots are traced between four images. For instance, the “12” in the first image (top) is traced to the “7” in the next image, which in turn is traced to the “16” in the third image, and finally the “3” in the last image (bottom). The traced dots form a candidate barcode (12-7-16-3) that corresponds to Aldh 18a1 (C). It is not necessary to have images generated from multiple rounds of hybridization before the dots are traced. For example, a series of images from a single round of hybridization with a fluorescent probe could be performed. The dots in the image can be traced as a sample changes over time, such as with displacement oligonucleotides.

In some embodiments, the dots are traced between images using a search radius (r). As an example, in FIG. 1, (B), the “12” has a search radius set at 10 pixels around it in the top image. In the next image, the “12” dot traces to the “7” dot because the “7” dot is within the search radius of the “12” dot. The process is iterated.

In some embodiments, wherein the search radius is between 1 and 100 pixels. In some embodiments, the search radius is between 1 and 90 pixels. In some embodiments, the search radius is between 1 and 80 pixels. In some embodiments, the search radius is between 1 and 70 pixels. In some embodiments, the search radius is between 1 and 60 pixels. In some embodiments, the search radius is between 1 and 50 pixels. In some embodiments, the search radius is between 1 and 40 pixels. In some embodiments, the search radius is between 1 and 30 pixels. In some embodiments, the search radius is between 1 and 20 pixels. In some embodiments, the search radius is between 1 and 10 pixels. In some embodiments, the search radius is between 1 and 5 pixels.

In some embodiments, the method of any of the previous embodiments, further comprises aligning each of the images using one or more reference positions in each image. In certain embodiments, the one or more reference positions are one or more dots at the same positions in each image.

In some embodiments, the method comprises fitting the dots in each of the images to a function that determines their spatial coordinates. In certain embodiments, the function is Gaussian or Airy.

In some embodiments, the spatial positions of the candidate barcodes can be determined by taking mean, median, or other functions, of the spatial positions of the dots that make up the candidate barcodes. In certain embodiments, this allows the candidate barcodes and their corresponding molecular targets to be localized to a high precision and resolved in dot dense images.

### Cost Functions

In some embodiments, the method comprises using a computer, assigning a cost to the plurality of candidate barcodes to obtain an assigned cost.

Cost minimization, as described here, is but one embodiment. There are other ways of performing optimization calculations, including other minimization functions, or even maximizing functions. For example, it is possible to perform maximization calculations, looking at the same set of criteria as with the cost function, but focusing on the number of used dots, rather than on the number of unused dots. It also may be possible to use a subset of the criteria described herein with respect to the cost minimization function.

In some embodiments, the method comprises assigning a cost, wherein the cost is an error function measuring the deviation of the observed pixel intensities from the pixel intensities predicted by trial solutions and is determined according to:

$$C = f ( {N}_{n d} )$$

wherein N_{nd} is a number of dots in the images that are not used in the candidate barcodes, and f is a function.

In some embodiments, the method comprises assigning a cost, wherein the cost is determined according to:

$$C = {\sum }_{b}^{B} ( {a}_{1} ( var ( {x}_{b} ) + var ( {y}_{b} ) ) + {a}_{2} var ( log ( {I}_{b} ) ) + {a}_{3} {δ}_{imperfect} ( b ) ) + {N}_{nd} .$$

where B is the set of all barcode candidates in the trial solution, b is a barcode candidate in B, and var (x_{b}), var (y_{b}), and var (log(I_{b})) are the variances of x-coordinates; y-coordinates and log intensities of each dot in the candidate barcode b; δ_{imperfect} is an indicator variable that is equal to 1 when b is an imperfect barcode and equal to 0 when it is a perfect barcode; a₁ is a position variance penalty; a₂ is a weight variance penalty; a₃ is a penalty for imperfect barcodes, are user set parameters; N_{nd} is a number of dots in the images that are not used in the candidate barcodes.

In some embodiments, the method comprises assigning a cost wherein the cost is determined using pixel intensities in the images and deviations from the pixel intensities predicted by the trial solutions.

In some embodiments, the method comprises assigning a cost, wherein the cost is determined using regularized regression to select candidate barcodes that account for dots in images.

### Pseudo-Color

A pseudo-color based barcoding scheme is developed to overcome limitations in the previous generation of the technology such as lack of visual signals that can be associated with the probes or small internal within cell when carrying out in situ experiments. See, for example, International Patent Application No. PCT/US2017/044994, FILED Aug. 1, 2017, and titled SEQUENTIAL PROBING OF MOLECULAR TARGETS BASED ON PSEUDO-COLOR BARCODES WITH EMBEDDED ERROR CORRECTION MECHANISM, the entire contents of which are herein incorporated by reference in its entirety for all purposes.

Although the term “pseudo-color” is used, one is not limited to using colors in this coding scheme. Symbols, letters, numbers, 2D barcodes, 3D barcodes, and combinations thereof can be used to uniquely identify molecular targets. As disclosed herein the number of pseudo-colors can far exceeds the number of actual colors that are associated with the detection probes used in the hybridization experiments.

In some embodiments, the method further comprises assigning dots a pseudo-color value or symbol in each image. In some embodiments, the method further comprises assigning a dot a pseudo-color value. In some embodiments, the method further comprises assigning each dot a pseudo-color value. In some embodiments, the method further comprises assigning each dot a symbol.

### Coding Schemes

The methods of the instant claims can utilize a variety of coding schemes. In some embodiments, the methods of the instant claims use a linear coding scheme. In some embodiments, the methods of the instant claims use a nonlinear coding scheme.

Linear coding schemes are error-correcting code schemes for which any linear combination of codewords is also a codeword. A nonlinear coding scheme utilizes a nonlinear relationship between codewords.

In some embodiments, a codeword is a sequence of symbols. In some embodiments, a symbol is part of a codeword. In some embodiments, codewords correspond to molecular targets. In some embodiments, a codeword in a codebook corresponds to an “empty” or “null” target. An “empty” or “null” target is not an actual molecular target and is used as a negative control.

In some embodiments, the symbol is an element of a codeword, wherein the codeword identifies the molecular target.

In some embodiments, the candidate barcodes are selected from linear codes.

In some certain embodiments, the linear codes are selected from the group consisting of BCH codes and Hamming codes. In certain embodiments, the linear code is a Reed-Solomon code.

In some embodiments, the method comprises identifying each candidate barcode using a coding scheme that does not have parity checks. In certain embodiments, a coding scheme that does not have parity checks is a nonlinear code.

In some embodiments, the method candidate barcodes are selected from nonlinear codes. In some certain embodiments, the nonlinear codes are selected from the group consisting of Reed-Muller codes, majority codes, and Golay codes.

### Dynamic Programming

Dynamic programming is a computer programming technique where an algorithmic problem is first broken down into sub-problems, the results are saved, and then the sub-problems are optimized to find the overall solution. This optimization involves finding the maximum and minimum range of the algorithmic query.

In some embodiments, the method comprises using dynamic programming to identify codepaths, wherein the codepaths are candidate barcodes.

In some embodiments, the method further comprises identifying candidate barcodes by summing syndromes.

### Sample and Molecular Targets

In some embodiments, the method comprises analyzing samples, wherein the samples comprise bacterial cells, archaeal cells, eukaryotic cells, or a combination thereof. In some embodiments, the samples comprise tissues, cells, or extracts from cells. In some embodiments, the samples comprise cells obtained from patients. In some embodiments, the samples comprise fluids obtained from patients.

In some embodiments, the sample comprises molecular targets that are selected from proteins, modified proteins, transcripts, RNA, DNA loci, exogenous proteins, exogenous nucleic acids, hormones, carbohydrates, small molecules, biologically active molecules, and combinations thereof. In some embodiments, the targets comprise subcellular features.

### Primary Probes

In some embodiments, the method comprises contacting a sample comprising a plurality of molecular targets with a plurality of one or more primary probes.

In some embodiments, the primary probe is selected from proteins, modified proteins, RNA, oligonucleotides, antibodies, antibody fragments, and combinations thereof.

In some embodiments, the primary probe comprises an oligonucleotide. In some embodiments, the detectably labelled probe comprises an oligonucleotide with a detectably moiety.

In some embodiments, the primary probe comprises oligonucleotides that are at least 5 nucleotides long. In some embodiments, the primary probe comprises oligonucleotides that are at least 6 nucleotides long. In some embodiments, the primary probe comprises oligonucleotides that are at least 7 nucleotides long. In some embodiments, the primary probe comprises oligonucleotides that are at least 8 nucleotides long. In some embodiments, the primary probe comprises oligonucleotides that are at least 9 nucleotides long. In some embodiments, the primary probe comprises oligonucleotides that are at least 10 nucleotides long. In some embodiments, the primary probe comprises oligonucleotides that are at least 11 nucleotides long. In some embodiments, the primary probe comprises oligonucleotides that are at least 12 nucleotides long. In some embodiments, the primary probe comprises oligonucleotides that are at least 13 nucleotides long. In some embodiments, the primary probe comprises oligonucleotides that are at least 14 nucleotides long. In some embodiments, the primary probe comprises oligonucleotides that are at least 15 nucleotides long. In some embodiments, the primary probe comprises oligonucleotides that are at least 16 nucleotides long. In some embodiments, the primary probe comprises oligonucleotides that are at least 17 nucleotides long. In some embodiments, the primary probe comprises oligonucleotides that are at least 18 nucleotides long. In some embodiments, the primary probe comprises oligonucleotides that are at least 19 nucleotides long. In some embodiments, the primary probe comprises oligonucleotides that are at least 20 nucleotides long. In some embodiments, the primary probe comprises oligonucleotides that are at least 21 nucleotides long. In some embodiments, the primary probe comprises oligonucleotides that are at least 22 nucleotides long. In some embodiments, the primary probe comprises oligonucleotides that are at least 23 nucleotides long. In some embodiments, the primary probe comprises oligonucleotides that are at least 24 nucleotides long. In some embodiments, the primary probe comprises oligonucleotides that are at least 25 nucleotides long. In some embodiments, the primary probe comprises oligonucleotides that are at least 26 nucleotides long. In some embodiments, the primary probe comprises oligonucleotides that are at least 27 nucleotides long. In some embodiments, the primary probe comprises oligonucleotides that are at least 28 nucleotides long. In some embodiments, the primary probe comprises oligonucleotides that are at least 29 nucleotides long. In some embodiments, the primary probe comprises oligonucleotides that are at least 30 nucleotides long. In some embodiments, the primary probes of any of the previous embodiments comprises oligonucleotides that are less than 35, 40, 45, 50, 100 nucleotides in length.

In some embodiments, the primary probe comprises a sequence that is complementary to the molecular target. In some embodiments the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the primary probe comprises one or more amplifier sequences. In some embodiments, the primary probe comprises two or more amplifier sequences. In some embodiments, the primary probe comprises three or more amplifier sequences. In some embodiments, the primary probe comprises four or more amplifier sequences. In some embodiments, the primary probe comprises five or more amplifier sequences. In some embodiments, the primary probe comprises six or more amplifier sequences. In some embodiments, the primary probe comprises seven or more amplifier sequences. In some embodiments, the primary probe comprises eight or more amplifier sequences.

In some embodiments, the one or more amplifier sequences are the same sequences. In some embodiments, at least one of the amplifier sequences in the one or more amplifier sequences are the same. In some embodiments, the one or more amplifier sequences are different from each other. In some embodiments, at least one of the amplifier sequences in the one or more amplifier sequences are different.

In some embodiments, the amplifier sequence comprises a nucleotide sequence that is at least 5 nucleotides long. In some embodiments, the amplifier sequence comprises a nucleotide sequence that is at least 6 nucleotides long. In some embodiments, the amplifier sequence comprises a nucleotide sequence that is at least 7 nucleotides long. In some embodiments, the amplifier sequence comprises a nucleotide sequence that is at least 8 nucleotides long. In some embodiments, the amplifier sequence comprises a nucleotide sequence that is at least 9 nucleotides long. In some embodiments, the amplifier sequence comprises a nucleotide sequence that is at least 10 nucleotides long. In some embodiments, the amplifier sequence comprises a nucleotide sequence that is at least 11 nucleotides long. In some embodiments, the amplifier sequence comprises a nucleotide sequence that is at least 12 nucleotides long. In some embodiments, the amplifier sequence comprises a nucleotide sequence that is at least 13 nucleotides long. In some embodiments, the amplifier sequence comprises a nucleotide sequence that is at least 14 nucleotides long. In some embodiments, the amplifier sequence comprises a nucleotide sequence that is at least 15 nucleotides long. In some embodiments, the amplifier sequence comprises a nucleotide sequence that is at least 16 nucleotides long. In some embodiments, the amplifier sequence comprises a nucleotide sequence that is at least 17 nucleotides long. In some embodiments, the amplifier sequence comprises a nucleotide sequence that is at least 18 nucleotides long. In some embodiments, the amplifier sequence comprises a nucleotide sequence that is at least 19 nucleotides long. In some embodiments, the amplifier sequence comprises a nucleotide sequence that is at least 20 nucleotides long. In some embodiments, the amplifier sequence comprises a nucleotide sequence that is at least 21 nucleotides long. In some embodiments, the amplifier sequence comprises a nucleotide sequence that is at least 22 nucleotides long. In some embodiments, the amplifier sequence comprises a nucleotide sequence that is at least 23 nucleotides long. In some embodiments, the amplifier sequence comprises a nucleotide sequence that is at least 24 nucleotides long. In some embodiments, the amplifier sequence comprises a nucleotide sequence that is at least 25 nucleotides long. In some embodiments, the amplifier sequence comprises a nucleotide sequence that is at least 26 nucleotides long. In some embodiments, the amplifier sequence comprises a nucleotide sequence that is at least 27 nucleotides long. In some embodiments, the amplifier sequence comprises a nucleotide sequence that is less than 35, 40, 45, 50, 100 nucleotides in length.

### Detectably Labelled Probes

In some embodiments, the method comprises barcoding molecular targets by using detectably labelled probes.

In some embodiments, the detectably labelled probe is selected from proteins, modified proteins, RNA, oligonucleotides, antibodies, antibody fragments, and combinations thereof. In some embodiments, the detectably labelled probe further comprises a detectably moiety. In some embodiments, the detectably moiety is a fluorophore.

In some embodiments, the detectably labelled probe comprises an oligonucleotide with a detectably moiety.

In some embodiments, the detectably labelled probe comprises oligonucleotides that are at least 5 nucleotides long. In some embodiments, the detectably labelled probe comprises oligonucleotides that are at least 6 nucleotides long. In some embodiments, the detectably labelled probe comprises oligonucleotides that are at least 7 nucleotides long. In some embodiments, the detectably labelled probe comprises oligonucleotides that are at least 8 nucleotides long. In some embodiments, the detectably labelled probe comprises oligonucleotides that are at least 9 nucleotides long. In some embodiments, the detectably labelled probe comprises oligonucleotides that are at least 10 nucleotides long. In some embodiments, the detectably labelled probe comprises oligonucleotides that are at least 11 nucleotides long. In some embodiments, the detectably labelled probe comprises oligonucleotides that are at least 12 nucleotides long. In some embodiments, the detectably labelled probe comprises oligonucleotides that are at least 13 nucleotides long. In some embodiments, the detectably labelled probe comprises oligonucleotides that are at least 14 nucleotides long. In some embodiments, the detectably labelled probe comprises oligonucleotides that are at least 15 nucleotides long. In some embodiments, the detectably labelled probe comprises oligonucleotides that are at least 16 nucleotides long. In some embodiments, the detectably labelled probe comprises oligonucleotides that are at least 17 nucleotides long. In some embodiments, the detectably labelled probe comprises oligonucleotides that are at least 18 nucleotides long. In some embodiments, the detectably labelled probe comprises oligonucleotides that are at least 19 nucleotides long. In some embodiments, the detectably labelled probe comprises oligonucleotides that are at least 20 nucleotides long. In some embodiments, the detectably labelled probe comprises oligonucleotides that are at least 21 nucleotides long. In some embodiments, the detectably labelled probe comprises oligonucleotides that are at least 22 nucleotides long. In some embodiments, the detectably labelled probe comprises oligonucleotides that are at least 23 nucleotides long. In some embodiments, the detectably labelled probe comprises oligonucleotides that are at least 24 nucleotides long. In some embodiments, the detectably labelled probe comprises oligonucleotides that are at least 25 nucleotides long. In some embodiments, the detectably labelled probe comprises oligonucleotides that are at least 26 nucleotides long. In some embodiments, the detectably labelled probe comprises oligonucleotides that are at least 27 nucleotides long. In some embodiments, the detectably labelled probe comprises oligonucleotides that are at least 28 nucleotides long. In some embodiments, the detectably labelled probe comprises oligonucleotides that are at least 29 nucleotides long. In some embodiments, the detectably labelled probe comprises oligonucleotides that are at least 30 nucleotides long. In some embodiments, the detectably labelled probes of any of the previous embodiments comprises oligonucleotides that are less than 35, 40, 45, 50, 100 nucleotides in length.

In some embodiments, the detectably labelled probe comprises a sequence that is complementary to the primary probe. In some embodiments, the sequence complementarity comprises at least 60%, 65%, 70%, 75%, 80%, 85%, 90%, 91%, 92%, 93%, 94%, 95%, 96%, 97%, 98%, 99%, or 100%.

In some embodiments, the detectably labelled probes comprise oligonucleotides with the same sequence. In some embodiments, the detectably labelled probes comprise oligonucleotides with different sequences

### Barcoding the Targets

In some embodiments, the method comprises barcoding one or more molecular targets. In some embodiments, the molecular targets that are selected from proteins, modified proteins, transcripts, RNA, DNA loci, exogenous proteins, exogenous nucleic acids, hormones, carbohydrates, small molecules, biologically active molecules, and combinations thereof. In some embodiments, the targets comprise subcellular features. For example, the nuclear lamin can be labeled with one set of barcodes, and nucleolus can be targeted with another set of barcodes. Then each sample can be uniquely labeled with a combination of barcodes on different subcellular compartments. In some embodiments, the method comprises barcoding targets, wherein the targets are different.

In some embodiments, the method comprises fluorescence detection. In some embodiments, the method comprises fluorescence detection or other methods of detection. In some embodiments, the method comprises sequential hybridization to detect target analytes.

In some embodiments, the probes are used in a method to barcode one or more molecular targets. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the probes are used in a method for linked amplification tethered with exponential radiance (LANTERN). See, for example, International Patent Application No. PCT/US2022/021826, FILED Mar. 24, 2022, and titled LINKED AMPLIFICATION TETHERED WITH EXPONENTIAL RADIANCE, the entire contents of which are herein incorporated by reference in its entirety for all purposes.

In some embodiments, the probes are used in ClampFISH experiments. See, for example, ClampFISH detects individual nucleic acid molecules using click chemistry-based amplification, Rouhanifard S.H. et al., Nature Biotechnology 37:84-89 (2019), the entire contents of which are herein incorporated by reference in its entirety for all purposes.

In some embodiments, the method comprises detectably labelled probes that are selected from proteins, modified proteins, RNA, oligonucleotides, antibodies, antibody fragments, and combinations thereof.

In some embodiments, the method comprises contacting each sample in the one or more samples with a first plurality of detectably labelled probes, so that the probes interact with one or more targets. In some embodiments, the method comprises imaging the sample after the first contacting step so that interaction of the detectably labelled probes with their targets is detected.

In some embodiments, the method comprises a contacting step that differs from another contacting step in the labelling of at least one of the targets.

In some embodiments, the method comprises a contacting step wherein each detectably labeled probe in the first plurality of probes is labelled with a detectably moiety.

In some embodiments, the method comprises a contacting step wherein each detectably labelled probe comprises a detectable moiety and at least one contacting step differs from another contacting step by having a different detectable moiety for each target.

In some embodiments, the method comprises a contacting step wherein at least two different detectably labelled probes that interact with a first target and wherein at least two different detectably labelled probes interact with a second target.

In some embodiments, the detectably labelled probes comprise labels selected from two, three, or four different labels.

In some embodiments, the barcode for the target in the sample comprises a signal that is amplified. In some embodiments, the barcode for the target in a sample comprises a signal that is amplified by rolling circle, padlock, branched DNA, ClampFISH, LANTERN, or any combination thereof.

In some embodiments, the method comprises using detectably labelled probes wherein each detectably labeled probe comprises the same detectable moiety and the same sequence.

In some embodiments, the method comprises detectably labelled probes wherein each detectably labelled probes interacts with its target through one or more intermediate probes each of which is hybridized to the target.

In some embodiments, the method comprises repeating the contacting and imaging steps, each time with a new plurality of detectably labelled probes so that a target in the sample is described by a barcode, and can be differentiated from another target in the sample by a difference in their barcodes.

In some embodiments, the method comprises an error correction round. See, for example, International Patent Application No. PCT/US2017/044994, FILED Aug. 1, 2017, and titled SEQUENTIAL PROBING OF MOLECULAR TARGETS BASED ON PSEUDO-COLOR BARCODES WITH EMBEDDED ERROR CORRECTION MECHANISM, the entire contents of which are herein incorporated by reference in its entirety for all purposes.

In some embodiments, the method of any of the previous embodiments further comprises an error correction step. In some embodiments, the error correction step comprises performing additional rounds of contacting and imaging prior or in between or after steps (i)-(v).

### Removing Probes

In some embodiments, the method comprises a step of removing the detectably labelled probes after one or more imaging steps. In some embodiments, the step of removing the detectably labelled probes comprises contacting the plurality of detectably labelled probes with an enzyme that digests a detectably labelled probes. In some embodiments, the step of removing comprises contacting the plurality of detectably labelled probes with a DNase, contacting the plurality of detectably labelled probes with an RNase, photobleaching, strand displacement, formamide wash, heat denaturation, or combinations thereof. In some embodiments, the step of removing comprises photobleaching to remove the detectably labelled probes.

In some embodiments, the method comprises removing detectably labelled probes by using stripping reagents, wash buffers, photobleaching, chemical bleaching, and any combinations thereof.

In some embodiments, the method comprises clearing the sample. In some embodiments the sample is cleared by CLARITY.

Certain techniques for removing probes are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

### Imaging the Sample

In some embodiments, the method comprises imaging the detectably labelled probes. In some embodiments, the method comprises imaging the barcodes. As understood by a person having ordinary skill in the art, different technologies can be used for the imaging steps.

In some embodiments, the imaging methods comprise but are not limited to epi-fluorescence microscopy, confocal microscopy, the different types of super-resolution microscopy (PALM/STORM, SSIM/GSD/STED), and light sheet microscopy (SPIM and etc.).

In some embodiments, the imaging methods comprise exemplary super resolution technologies include, but are not limited to I⁵M and 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. For examples: Chi, 2009 “Super-resolution microscopy: breaking the limits,” Nature Methods 6(1): 15-18; Blow 2008, “New ways to see a smaller world,” Nature 456:825-828; Hell, et al., 2007, “Far-Field Optical Nanoscopy,” Science 316:1153; R. Heintzmann and G. Ficz, 2006, “Breaking the resolution limit in light microscopy,” Briefings in Functional Genomics and Proteomics 5(4):289-301; Garini et al., 2005, “From micro to nano: recent advances in high-resolution microscopy,” Current Opinion in Biotechnology 16:3-12; and Bewersdorf et al., 2006, “Comparison of I⁵M and 4Pi-microscopy,” 222(2): 105-1 17; and Wells, 2004, “Man the Nanoscopes,” JCB 164(3):337-340.

In some embodiments, electron microscopes (EM) are used for imaging.

In some embodiments, an imaging step detects a target. In some embodiments, an imaging step localizes a target. In some embodiments, an imaging step provides three-dimensional spatial information of a target. In some embodiments, an imaging step quantifies a target. By using multiple contacting and imaging steps, provided methods are capable of providing spatial and/or quantitative information for a large number of targets in surprisingly high throughput. For example, when using F detectably different types of labels, spatial and/or quantitative information of up to FN targets can be obtained after N contacting and imaging steps.

Certain techniques for imaging are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises analyzing cell size and shape, markers, immunofluorescence measurements, or any combinations thereof.

### Flurophores

In some embodiments, the method comprises detecting the probes, detectably labelled probes, or oligonucleotides thereof with fluorophores. In some embodiments, the detectably labelled probe comprises a fluorophore.

In some embodiments, the fluorophore is any fluorophore deemed suitable by those of skill in the arts.

In some embodiments, the fluorophores include but are not limited to fluorescein, rhodamine, Alexa Fluors, DyLight fluors, ATTO Dyes, or any analogs or derivatives thereof. In some embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the fluorphores include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl)naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium(II), Texas Red, TSQ, Umbelliferone, or Yellow fluorescent protein.

In some embodiments, the fluorophores include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the fluorophores comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the fluorophore comprises a nanomaterial. In some embodiments, the fluorophore is a nanoparticle. In some embodiments, the fluorophore is or comprises a quantum dot. In some embodiments, the fluorophore is a quantum dot. In some embodiments, the fluorophore comprises a quantum dot. In some embodiments, the fluorophore is or comprises a gold nanoparticle. In some embodiments, the fluorophore is a gold nanoparticle. In some embodiments, the fluorophore comprises a gold nanoparticle.

### Washes

In some embodiments, the method of any of the preceding embodiments, comprises optionally washing the sample after each step. In some embodiments, the sample is washed with a buffer that removes non-specific hybridization reactions. In some embodiments, formamide is used in the wash step. In some embodiments, the wash buffer is stringent. In some embodiments, the wash buffer comprises 10% formamide, 2×SSC, and 0.1% triton X-100s.

Having described the embodiments in detail, it will be apparent that modifications, variations, and equivalent embodiments are possible without departing the scope of the defined in the appended claims. Furthermore, it should be appreciated that all examples in the present disclosure are provided as non-limiting examples.

The following non-limiting methods and examples are provided to further illustrate the embodiments disclosed herein. It should be appreciated by those of skill in the art that the techniques disclosed in the methods and examples that follow represent approaches that have been found to function well in practice, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the embodiments.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### METHODS

### Method 1

### An Explanation of the Error-Correcting Codes in Imaging-Based Spatial Genomics

Linear error correcting-codes use codewords that are sequences of symbols. Codewords can be divided into the information symbols, which alone represent all of the information encoded in a codeword, and the parity check symbols, which add redundancy and robustness to error to the codeword. Parity check symbols can be calculated as linear combinations of the information symbols. The number of symbols is denoted as n, the number of information symbols as k, the size of the alphabet from which the symbols are drawn as q, and the number of parity check symbols as n−k. The number of non-zeros in a codeword is called the weight (w) of the codeword. Traditionally, some ISG experiments have used q-ary parity check codes with n=4 and k=3 or n=5 with k=3 or 4 where all symbols in the alphabet including zero are represented by a pseudo-color. MERFISH uses weight-4 codewords from binary error-correcting codes (q=2) where 0s are represented by absence of dots and are not probed. The barcoding blocks of binary codes consist of a single image whose dots take on the value of a single pseudo-color. MERFISH has used Hamming codes (Chen et al.) and non-linear codes (Xia et al.).

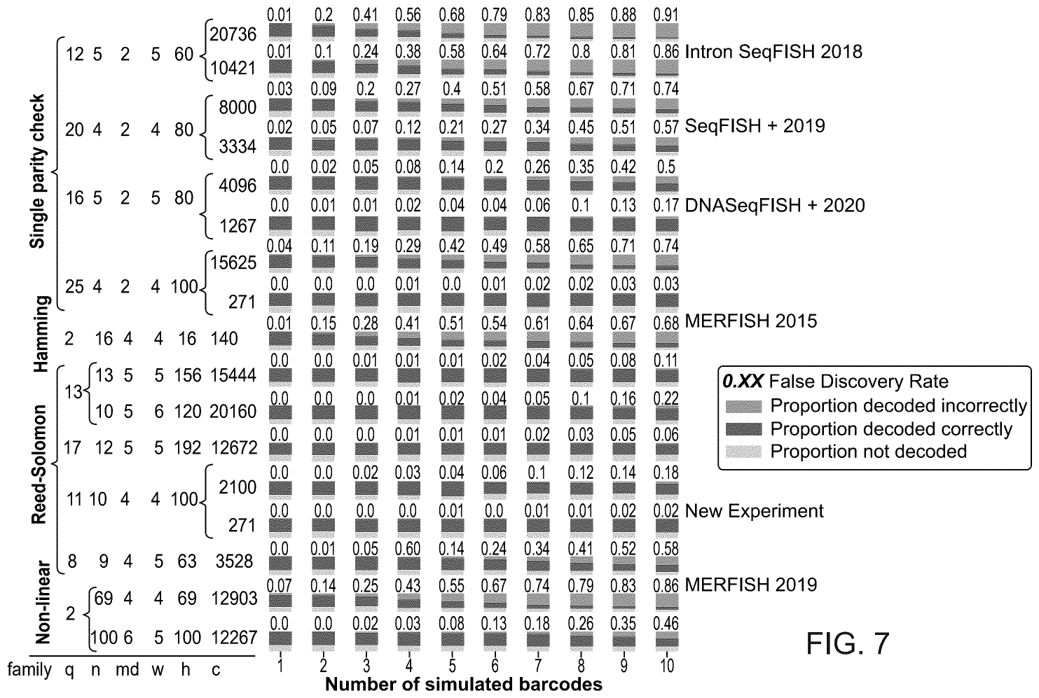

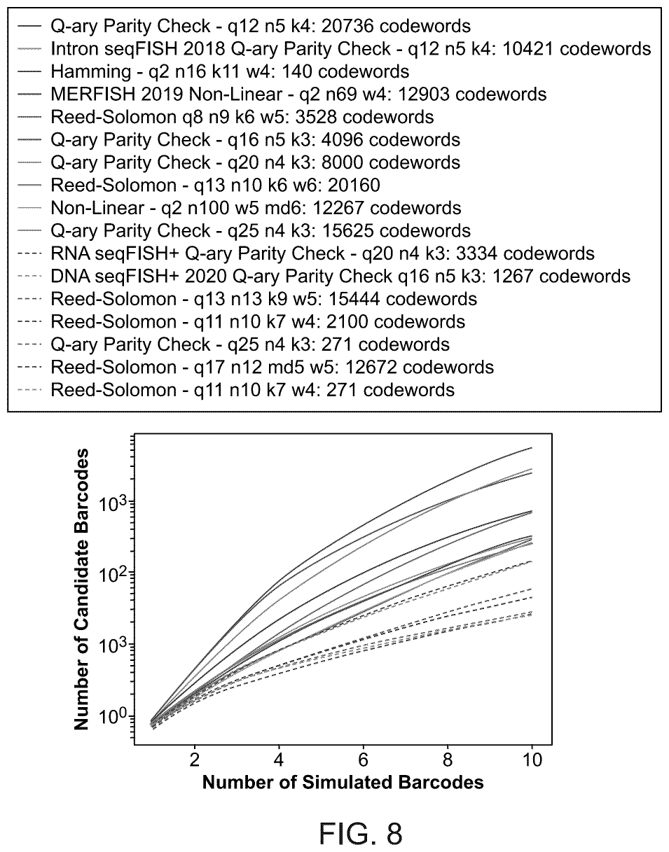

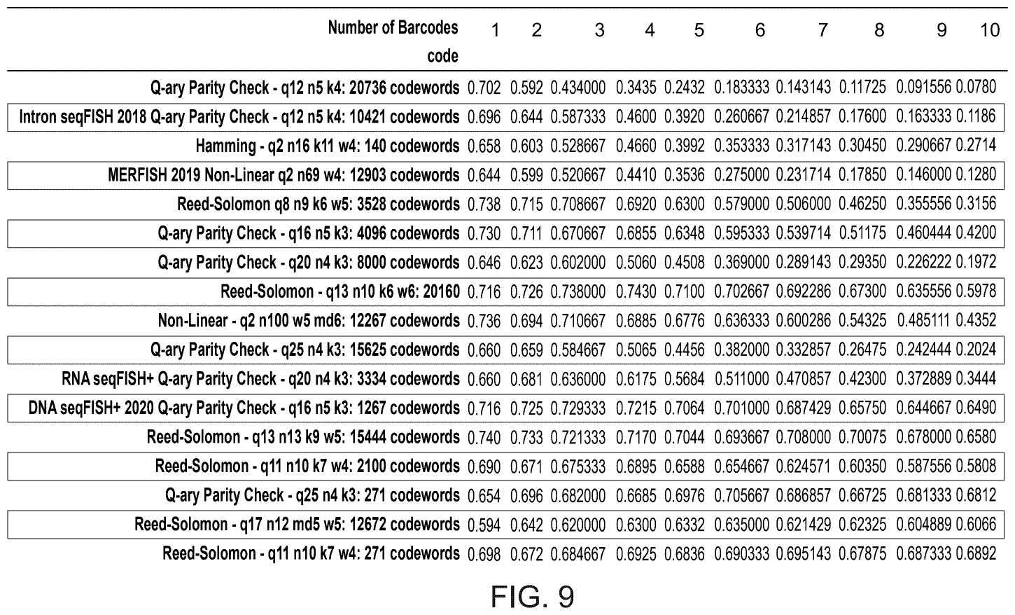

One measure of a code's robustness is its minimum Hamming distance, the minimum Hamming distance between any pair of its codewords. Reed-Solomon codes are a part of a special class of linear error-correcting codes called maximum distance separable codes for which md=n−k+1. There are ways to increase the robustness of a code in ways that are not measured by the minimum Hamming distance. For example, Reed-Solomon codes use symbols from alphabets with power of a prime number as their size. Using barcoding block sizes of greater than one pseudo-color as in the methods' implementation of q-ary parity check codes and Reed-Solomon codes adds an extra rule that reduces ambiguity in superimposed barcodes-each barcode can only have one value or one dot in each barcoding block. Increasing the average Hamming distance between codewords without increasing the minimum Hamming distance of a code can also improve robustness. Thinning a codebook to not include all available codewords or adding parity checks that increase the average, but not minimum Hamming distance, can also increase the robustness of codes (FIG. 7, FIG. 8, FIG. 9).

The minimum Hamming distance of linear error-correcting codes is bounded by: Minimum weight of codeword<=minimum Hamming distance

However non-linear error-correcting codes are not subject to this bound, which may provide an opportunity to more efficiently and robustly encode information at the cost of more computationally expensive decoding. To test the potential of non-linear error-correcting codes, a non-linear error-correcting code was designed. By first choosing a minimum Hamming distance of 6, iterating over all combinations of 100 choose 5, and then adding any combination to the codebook that is at least 6 Hamming distance away from any combination that had previously been added (FIG. 7, FIG. 8, FIG. 9).

### EXAMPLES

### Example 1

### Reed-Solomon Codes

Reed-Solomon codes were used in an exemplary example for encoding and decoding signals from barcodes in a plurality of molecular targets from images obtained from a ISG experiment.

Reed-Solomon codes are block codes whose codewords are composed of symbols drawn from finite fields. The number of symbols, n, in each codeword can be up to one greater than the order of the finite field from which the symbols are drawn. All finite fields contain a zero element, and the weight, w, of a codeword is defined as the number of non-zero symbols that it contains (Lin and Costello). The number of codewords of any given w in any MDS code, including Reed-Solomon codes, is theoretically determined (Macwilliams and Sloan).

Encoded ISG barcodes using Reed-Solomon codes were made in a ‘one-hot’ manner. Each symbol, drawn from a finite field of order q, were represented by a barcoding block of q−1 read-out hybridization images. Non-zero symbols were read out in one image of each barcoding block, appeared as a dot in the image, and zero symbols were not read, remaining dark in all images of the barcoding block. Thus, the number of times that each ISG barcode must be probed by only using barcodes of low weights were limited.

### Example 2

### Fitting Dots with ADCG

Dense ISG images have overlapping dots which are difficult to distinguish using Laplacian of Gaussians peak finding, the method used in most prior works (Raj et al.; Lubeck et al.; Shah et al.). To better distinguish these dots, an algorithm was adapted that excelled in a benchmarking study for processing dense 2D single molecule localization microscopy (SMLM) images, Alternating Decent Conditional Gradient (ADCG) to process ISG image stacks (Sage et al.; Boyd et al., Schiebinger, and Recht).

ADCG fits models of images as superpositions of Gaussian point spread functions (PSFs) emitted by fluorescent molecules,

$${E}_{xy} = {\sum }_{k = 1}^{n} {b}_{k} {e}^{\frac{{( x - {x}_{k} )}^{2} + {( y - {y}_{k} )}^{2}}{{σ}_{k}^{2}}} δ ( \vert x - {x}_{k} \vert < 3 {σ}_{max} ) δ ( \vert y - {y}_{k} \vert < 3 {σ}_{max} ) , . \qquad Equation 1$$

where E_{xy} denotes the model's expected intensity of the pixel in the x^{th} column and y^{th} row in the image; xₖ, yₖ, bₖ, and σₖ are the x-coordinate, y-coordinate, brightness and width parameters of the k^{th} PSF in the model; δ represents an indicator function that saves computation time by not calculating the PSF tails; and σₘᵢₙ and σₘₐₓ bound allowed values of σₖ. The width is included as a fit parameter because FISH dots may vary in size due to their distance from the imaging plane and because they are generated by multiple dye molecules attached to an RNA strand of finite size and varying conformation. The fitting procedure minimizes a least squares loss function,

$$Loss = {\sum }_{xy} {( {0}_{xy} - {E}_{xy} )}^{2} , . \qquad Equation 2$$

where σ_{xy} denotes the observed intensity of the xy^{th} voxel in the image.

ADCG iteratively fits models by adding one new PSF at a time. The first step of each iteration is to approximate the location of the next PSF to add to the model by convolving the image-model residuals with a 2-D Gaussian of width σ_{xy,min} and σ_{z,min} then finding the local maxima. Each PSF's width is approximated by grid search where linear least squares fits find associated brightnesses. Second, gradient descents refine the coordinate then the width and the brightness estimates. The indicator functions are not differentiated when calculating gradients of the loss, but retain them in the gradient function to save calculation time. Third, repeated gradient descent on the coordinates then brightness and width of all PSFs adjusts the parameters of all PSFs in the model until the loss converges or for a maximum number of iterations. Finally, PSFs with brightness below the minimum allowed are removed. PSFs are added to the model until no new PSFs with brightness larger than the minimum allowed are found, the improvement in the objective function is below a threshold, or until a maximum number of PSFs have been added to the model. When the algorithm terminates because the brightness or objective change is below a threshold, it discards the changes from the final iteration to avoid overfitting. For computational efficiency, images are broken up images into overlapping tiles, run the fit on each tile, and then piece the results from each tile together for the full image, removing duplicate PSFs found in the overlapping regions.

### Example 2

### Fiducial Marker Matching Registration

Dots found in different barcoding blocks of ISG experiments must be aligned to correct for drift over the course of the experiment and recognize which were read out from the same barcode. Previous studies (Lubeck et al.; K. H. Chen et al.; Shah et al.; Moffitt et al.) have aligned hybridization images using phase-cross correlation (Guizar-Sicairos, Thurman, and Fienup) on independent images of either DAPI stains or fiducial markers acquired concurrently but at a different wavelength than the readout images. This approach introduces errors from optical aberration, differences in light path alignment, and movement of the objective in between scans. These sources of error are reduced by aligning the dots using diffraction limited objects as fiducial markers in the readout images identified with a pattern matching algorithm. Images are registered in the same channel as each other using translations and images in different channels using affine transformations.

Fiducial markers do not move relative to the slide, so they appear in a fixed pattern that drifts with the field of view in the readout hybridizations. The algorithm searches for the dots in a fiducial marker pattern found in a reference image of only the fiducial markers among all dots found in readout images. The fiducial marker pattern is identified by matching the position vectors that separate pairs of fiducial markers. Each matching pair of separation vectors in the reference and readout images votes to classify the dots on their ends as matches. This is similar to another pattern matching algorithm that matches triangles, rather than vectors (Groth et al.).

The step is to find a first matching pair of separation vectors. This begins by assembling a list of the most distant pairs of fiducial markers from the reference image, excluding any fiducial marker that is within the allowed matching error distance, ε, of another fiducial marker. Two fiducial markers are denoted in a pair as u and v such that the x-component, uₓ, of u's position vector, u, is less than the x-component, vₓ, of v's position vector, v. Initially searching for the most distant pairs of fiducial markers lets us narrow the region of the readout image where a search must be conducted for u and v to around the corners. More dots are ruled out by setting bounds on the brightness ratio between fiducial markers in the reference image and readout image. When drifts are small, the search can be restricted to dots within a maximum allowed shift between images.

To find an initial match, the lists of dots are sorted in the readout image that may match to u and v by their x coordinate from lowest to highest. Individual candidate dots u′ and v′ are denoted. First, the x-components of the vectors are compared separating each u′ and v′, v′−u′, with the vector separating reference dots, v−u. The method starts with the first u and v candidates then proceed through the following logical flow: if (vₓ′−uₓ′)−(vₓ−uₓ)>ε, then v′ cannot possibly match v, so it is discarded, and the method starts again at the beginning of the revised lists. Likewise, if (vₓ′−uₓ′)−(vₓ−uₓ)<−ε, then u′ cannot possibly match u, so it is discarded, and the method starts at the beginning of the revised lists. If ∥(v′−u′)−(v−u)∥>ε, then the next v′ is checked in the list. If ∥(v′−u′)−(v−u)∥<ε, the separation vectors match and a search proceeds for the rest of the fiducial markers relative to it. When there is fewer than one candidate dot for every error window of 2ε, this algorithm will find matches in O(n) time.

To begin the search for the rest of the fiducial markers in the readout image, a queue is initialized containing the first two candidate fiducial marker matches, (u,u′) and (v,v′). The method then begins taking fiducial marker matched dots from the queue and searching for other fiducial markers from the reference pattern at their known relative position from the already matched dot in the readout image with a KDTree. For example, if the dot α′ is already matched to the fiducial marker α, a search is made for another dot β′ that matches another fiducial marker β such that ∥β′−(α′+(β−α))∥<ε. If such a β′ is found, the candidate-reference pair, (β, β′), is added to the candidate match queue. Each (β, β′) candidate match that is found relative to an (α, α′) candidate match is a matching edge that votes for the (α, α′) and (β, β′) matches. Once an (α, α′) candidate matching pair has received ten votes from matching separation vectors, the matched is confirmed, then the algorithm proceeds to search for matches with the next (α, α′) candidate-reference pair in the queue. If no (β, β′) candidate matches are found in the first ten searches from a given (α, α′) pair of candidate matches, the (α, α′) candidate match is discarded. If fewer than five matches have been confirmed with 10 matching edge votes after exhaustively searching from initial (u,u′) and (v,v′) candidate matches, all matches are discarded and the algorithm searches for new initial matches.

To set search parameters, the algorithm first grid searches for parameters to match the fiducial markers in two reference images of only the fiducial markers acquired at the beginning and end of the experiment. The strictest values of the search radius are used that allow the algorithm to find 90% of the matches that the least strict parameters found. The minimum brightness considered for a match to an initial dot is 50% less than the ratio of the greatest reduction in brightness from the initial to final reference image. The maximum allowed brightness ratio of matches to initial fiducial markers is the maximum observed brightness ratio of initial to final reference dot.

### Example 3

### ISG Representation of Error-Correcting Codes

Reed-Solomon codes were used to encode the experiment. Reed-Solomon codes are a family of error-correcting codes with an elegant mathematical construction wherein the maximum distance separable: the minimum Hamming distance, between any two codewords in the error-correcting code, is the maximum possible for a linear error-correcting code given the number of parity check symbols it contains.

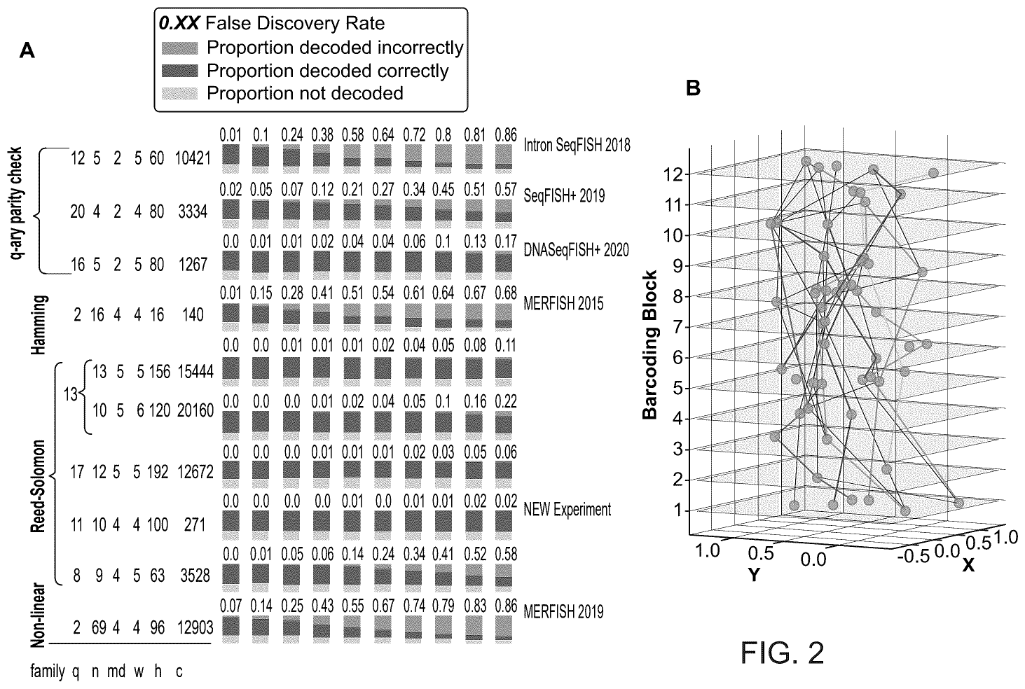

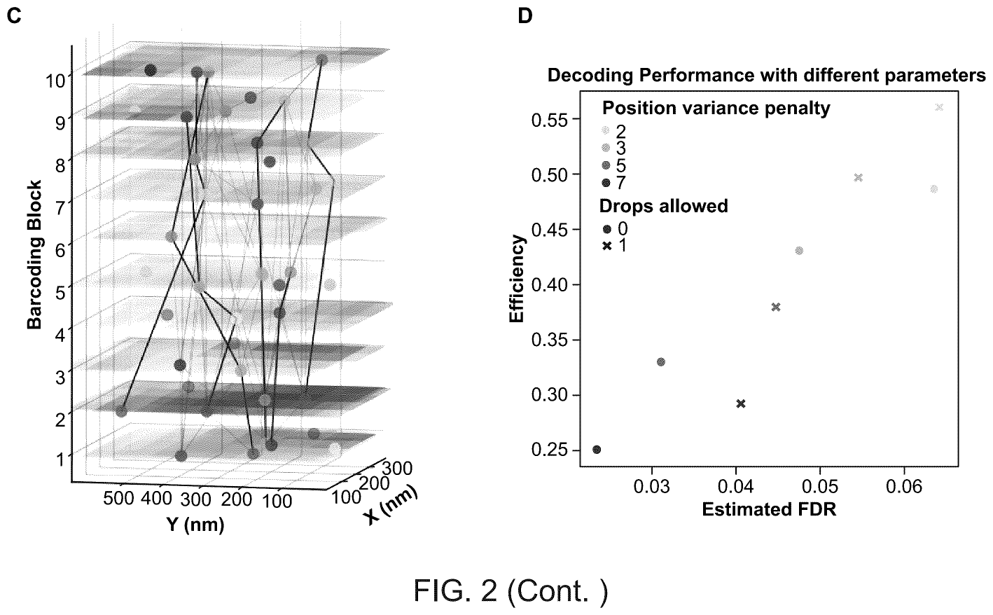

To represent Reed-Solomon codes using ISG, codewords were used with a fixed weight (number of non-zero symbols) and probe only non-zero symbols in each codeword. Barcoding blocks with no dot represent a zero symbol. The experiment was encoded using weight 4 codewords from the Reed-Solomon code with 7 information symbols and 3 parity check symbols from an alphabet of size 11. The simulations include codewords from other Reed-Solomon codes with weights of 4, 5, and 6 (FIG. 2, FIG. 7).

### Example 4

### Generating Codebooks and Parity Check Matrices

Reed-Solomon codes are defined using the abstract algebra structures of polynomial rings over finite fields. An equivalent representation to the generator polynomial is a generator matrix with elements from a finite field (Lin and Costello). To compute the codewords with the desired number of non-zeros, the systematic form (row reduced echelon form) of the generator matrix was found, which computes codewords that have parity check symbols appended to the information symbols in the message.

$$m G = c \text{,.} \qquad Equation 3$$

$$G = [ I \vert P ] \text{,.} \qquad Equation 4$$

The generator matrix in systematic form consists of an identity matrix with parity check elements concatenated. This allowed consideration of only messages with the desired number of non-zeros or fewer, compute the parity check symbols for the message, then discarded codewords that have more than the desired number of non-zeros A check was performed that verifies that the same number of codewords as predicted by the theoretical weight enumeration formula was found (MacWilliams and Sloan).

### Example 5

### Assigning Genes to Codewords

The assignment of genes to codewords was optimized to minimize the maximum sum FPKM expression of all genes probed in any single hybridization image. Alignment across channels is worse than alignment within a single channel, so after assigning genes to codewords, barcoding block and pseudo-color values were assigned to hybridization and channel images to maximize the number of barcodes that are probed in the same channel either 3 out of 4 or 4 out of 4 times.

### Example 6

### SeqFISH+ 2019 Image Processing Workflow

The snakemake workflow manager (Mölder et al.) was used to define a reproducible workflow for processing ISG images. The seqFISH+ 2019 experimental data is divided into sub-experiments in each of the three channels encoding each 3333 or 3334 genes expressing at approximately 33,333 FPKM over 80 hybridizations divided into four barcoding blocks of 20 pseudo-colors. This design avoided error from cross channel image registration and reduced ambiguity by partitioning barcodes that may have overlapped into independent subdivisions. The workflow first fits fluorescent beads used as fiducial markers with ADCG in all images, then finds the translations of readout images relative to a bead only reference image of the same channel by matching fiducial marker patterns. Before fitting dimmer FISH dots, the workflow removes the fiducial markers and autofluorescence from readout images by subtracting aligned reference images from them. It then subtracts the scattering background intensity estimated by applying a median filter with a 5×5 pixel kernel and then using the rolling ball algorithm with a radius of 3.3 pixels (Sternberg et al.; van der Walt et al.) and masks out intensity from pixels between cells. The same hand drawn masks used in the original study are used (Eng et al.). The workflow then finds FISH dots in the preprocessed images with ADCG, aligns their fit coordinates, splits them into groups aligning to each cell mask, and then uses syndrome decoding to infer which barcodes generated the PSFs in each cell. Only complete barcodes are allowed.

### Example 7

### Cross-Channel Reed-Solomon Encoded ISG Workflow

A Reed-Solomon encoded experiment used 100 images in three channels and 34 hybridizations to represent ten barcoding blocks of ten pseudo-colors. Segmentations were drawn manually with Cellpose from FISH images of cells hybridized with poly-T probes. First, beads used as fiducial markers were fit in images where cells were masked out by segmentation masks. Reference images containing only the beads and no readout probes were matched between channels and to beads found in images with readouts in the same channel. The channel with the most matching fiducial markers from the readout images to its reference image was used as the reference channel for each position. Images in the reference channel were registered to their reference image by translation, and images in the other channels were registered by affine transformation to the reference channel image. Background subtraction for images in each channel was performed as in the 2019 seqFISH+ experiment processing workflow. For this experiment, the increased minimum distance allows incomplete barcodes with only 3 dots (1 missing dot) and the missing dot penalty (a3 in equation 12) is set to 2.

### Example 8

### Simulations to Determine Density Robustness of Codes

Imageless simulations of decoding performance with various codes included the standard q-ary parity check codes, the Hamming code in the original MERFISH (Chen et al.), the non-linear cover code used in later MERFISH (Xia et al.), Reed-Solomon Codes, and a new non-linear code. First, truths were simulated for each replicate of each condition for each code. x and y coordinates of each object were drawn from a uniform distribution from 0 to 1. The codeword of each object was randomly selected from the given codebook with equal probability for each codeword. Then, observed dot locations simulated from the truths that included readouts of each simulated transcript in respective pseudo-color-barcoding blocks. The localization error from the simulated true location of each object was drawn from a 2D normal distribution with sigma=0.5. Each readout had a 1/20 probability of being dropped in the simulation data. Each simulated image has a 1/50 probability of having a non-specific dot with x and y coordinates drawn from a uniform distribution from 0 to 1. Simulated dots were fed into the SeqFISHSyndromeDecoding.jl package with search radius of 10, lateral variance penalty of 10, and no drops allowed.

### Example 9

### Dynamic Programming was Used to Identify All Sets of Aligning Dots that Could Represent a Barcode

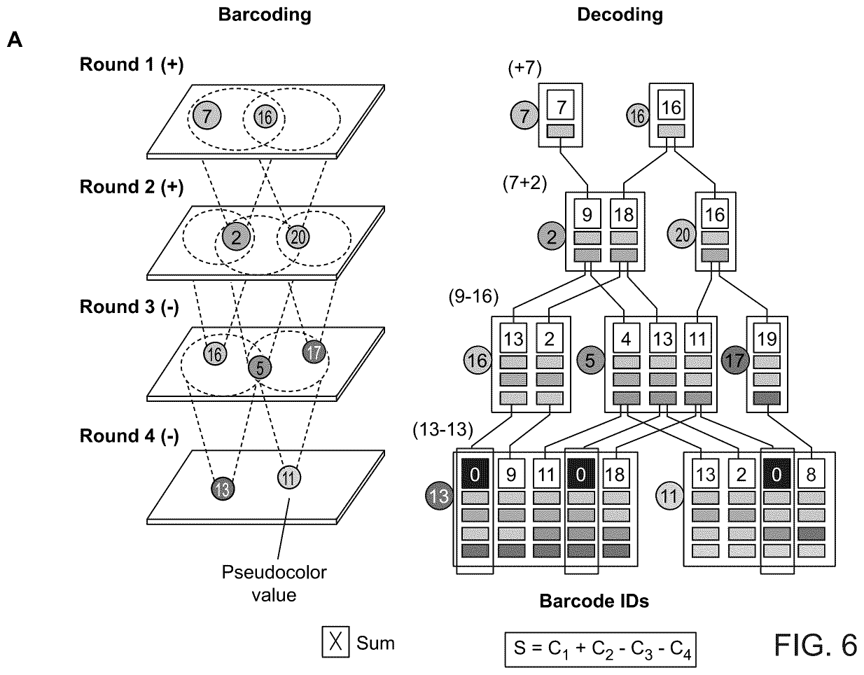

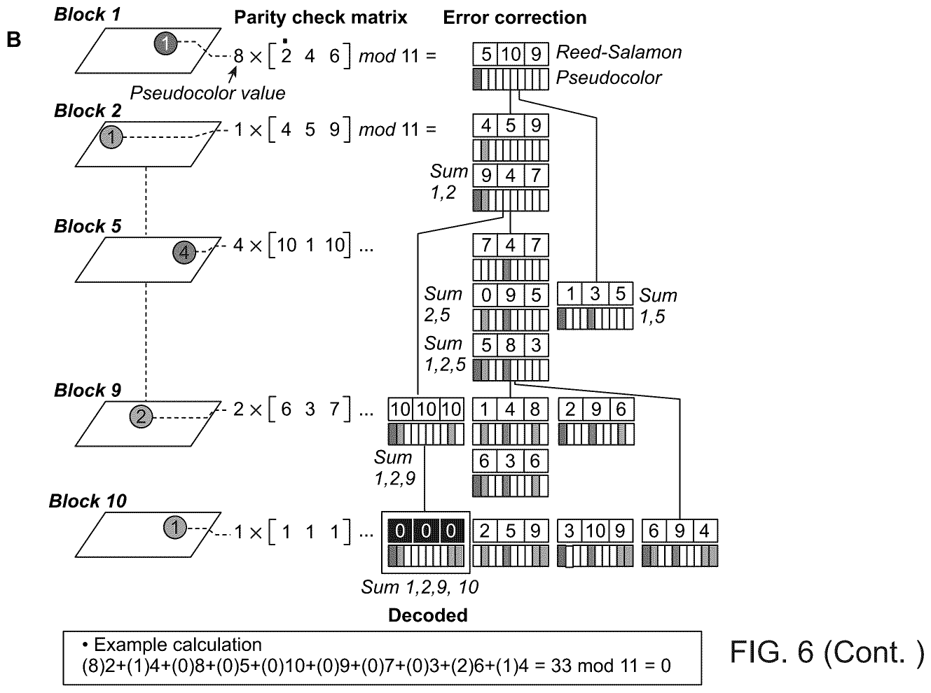

After preprocessing the images, and fitting dots, dynamic programming was used to identify all sets of aligning dots that could represent a barcode (FIG. 6). In dense datasets, recombinations of dots from two or more overlapping barcodes and dots originating from non-specific binding events give rise to spurious solutions (FIG. 1). Integer linear programming was used to choose the best decoding solution with the constraint that no two barcodes that contain a shared dot are allowed in a solution. Each solution is given a cost proportional to the sum of the position variances and log brightness variances of dots comprising each barcode, and the number of dots in the conflict network that are not decoded in the solution (FIG. 1).

This decoding method was applied to published seqFISH+ data probing mRNAs of 10,000 genes in NIH 3T3 cells (Eng et al.). The efficiency of the decoding method was measured when using a range of decoding cost parameters by regressing the average transcript counts of 60 genes it found in 225 NIH 3T3 cells with each parameter set against the average transcript count of the same genes previously found by smFISH in 288 other cells taken from the same culture (FIG. 1). The processing approach achieves greater efficiency with lower estimated false discovery rate (FDR) than previously reported.

Next, a comparison was performed with different error-correcting codes used in the ISG literature and new error-correcting codes of the instant methods to resolve overlaps in simulated dense samples to demonstrate that strong codes produce fewer spurious solutions when decoding superimposed barcodes in ISG images. To measure the robustness of various codes to ambiguity arising from overlaps, dot locations were simulated from multiple overlapping barcodes and evaluated decoding results for each code (FIG. 2, FIG. 7, FIG. 8). Codes whose codewords differ more from one another were found to be more robust to density. The minimum Hamming distance of a code and average Hamming distance are measures of how much codewords within a code differ from one another. Using barcoding blocks with many pseudo-colors rather than barcoding blocks with a single pseudo-color (binary codes) also increases robustness because only one dot from any given barcoding block is allowed in any barcode. Thinning codebooks to reduce the number of barcodes under consideration also reduces the number of ways to recombine dots into spurious barcodes (FIG. 9). The simulations showed that using stronger codes, the methods were able to disentangle many overlapping barcodes at densities where most codes used in the literature would incorrectly decode almost all barcodes (FIG. 2).

To validate the simulation results, a ISG experiment was designed by using an encoded Reed-Solomon code (FIG. 2). The experiment probed genes expressed at a sum total of 50,000 FPKM in bulk RNA sequencing and imaged 4 readouts from each transcript over 100 images in 34 hybridizations and 3 optical channels. This is in contrast to the previous 10,000 gene seqFISH+ experiment which consisted of 3 separately encoded sub-experiments each probing 3333 or 3334 genes expressed at a total of ˜33,333 FPKM over 80 hybridizations in a single channel. The new experiment probed genes expressed at 67% higher FPKM for a single code. Even with this greater density, a good efficiency relative to smFISH for 21 selected genes measured in cells taken from the same culture and estimated FDR lower than for the previous 10,000 gene seqFISH experiment both as originally reported and with reprocessing.

### Example 10

### Error Correcting Code Used for seqFISH+

The methods for identifying candidate barcodes from dot locations extracted from registered and preprocessed images will work for any experiments encoded using linear block code, which includes Reed-Solomon codes.

The methods were applied to the simpler error correcting code used for seqFISH+ in (Eng et al.). The first step was to find all aligning dots that could have been readout from a valid barcode. Dynamic programming was used to find aligning dots across barcoding blocks that satisfy the error-correcting code's parity check equations (FIG. 1, FIG. 6). The dynamic programming algorithm allowed identification of candidate barcodes with exponential complexity in density rather than combinatorial complexity in the density. The original seqFISH+ experiment encoded transcripts using four barcoding blocks. All possible combinations of pseudo-colors were allowed in the first three barcoding blocks, but the final pseudo-color was determined by the parity check equation:

$${C}_{4} = {C}_{1} + {C}_{2} - {C}_{3} ; \qquad Equation 5$$

where cᵢ denotes the pseudo-color of a barcode in barcoding round i, and addition and subtraction operations are modulo 20, the number of pseudo-colors. The parity check equation defines the syndrome,

$$s = {C}_{1} + {C}_{2} - {C}_{3} - {C}_{4} ; \qquad Equation 6$$

such that it will be 0 if and only if the dots' pseudo-colors represent a codeword. This method often identified networks of conflicting candidate barcodes (FIG. 1). Conflicts were resolved by scoring each feasible solution with an objective function, then using integer linear programming to choose the optimal solution (FIG. 1). More generally, when encoding a seqFISH experiment using any linear block code such as a Reed-Solomon code, the parity check equations can be written as a matrix equation where the syndrome is a vector all of whose elements will be zero if and only if the dots' barcoding blocks and pseudo-colors together form a valid codeword. To calculate a general syndrome with vectors of symbols from each barcoding block, which includes both pseudo-colors read out from a barcode in some barcoding blocks zeros in other barcoding blocks for which the barcode is not probed the equation was used:

$$s = H c ; \qquad Equation 7$$

where H is the parity check matrix of the error-correcting code and c is the vector of symbols from each barcoding block. Errors can be corrected when one or two readout dots are undetected by either using a standard error-correction algorithm, such as the Berlekamp algorithm or BKTree search, to infer which dots are missing and determine which barcode the detected dots are closest to.

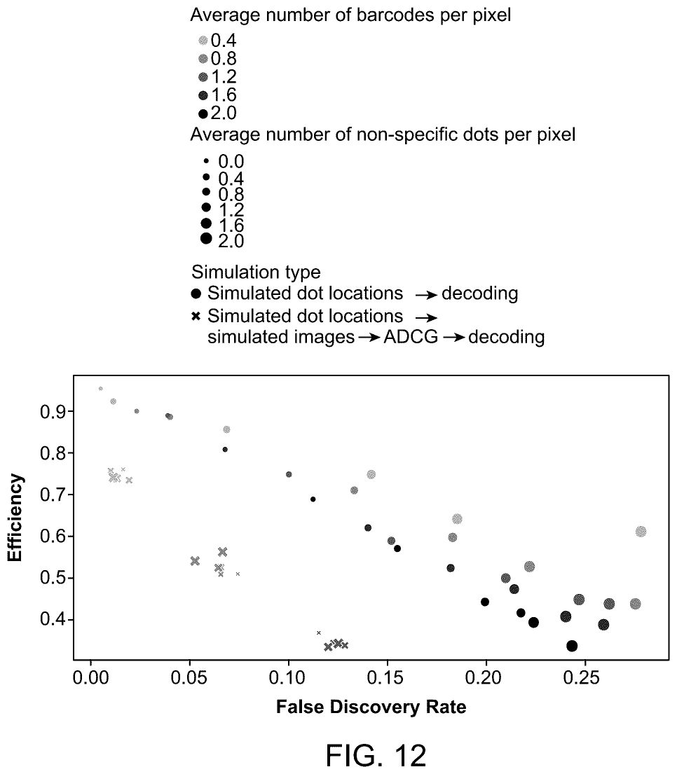

The method was demonstrated by decoding seqFISH+ data (Eng et al.) and measuring its decoding efficiency with a range of cost parameters (equation 12) relative to smFISH. This was done by regressing the average transcript counts of 60 genes it found in 225 NIH3T3 cells against the average transcript count of the same genes previously found by smFISH in 288 other cells taken from the same culture (FIG. 1). The processing approach achieved greater efficiency with lower False Discovery Rate (FDR) than previously reported (Eng et al.). To further investigate how the methods performed with the (Eng et al.) error correcting code and predict how they will perform with experiments encoded with Reed-Solomon codes, a simulation was made with image and dot location data with various barcode and nonspecific dot densities, and then processing the data by fitting the images with ADCG (Boyd et al.), and then running syndrome decoding on both the directly simulated and ADCG recovered dot locations (details in methods). In the simulations with the (Eng et al.) error correcting code (FIG. 12), it was found that ADCG, the dot detection algorithm used, is more sensitive to density than syndrome decoding. This is because overlapping dots in an image cannot be distinguished, but can introduce localization errors to the dots that are detected. With low numbers of non-specific dots, the imageless simulated decoding performed well at densities of up to 2 barcodes/pixel. The estimated FDR in the simulations was higher than true FDR, suggesting that the FDR estimator used for real data is conservative.

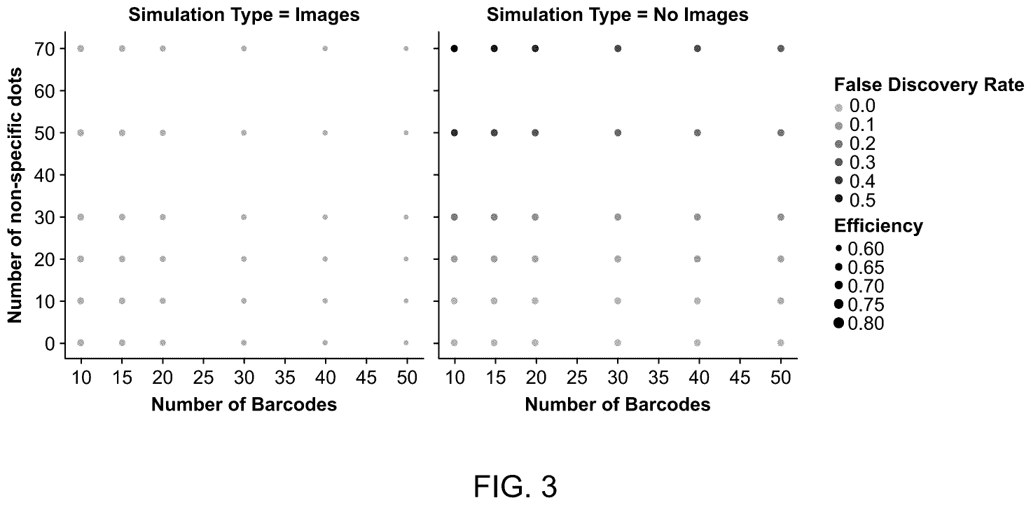

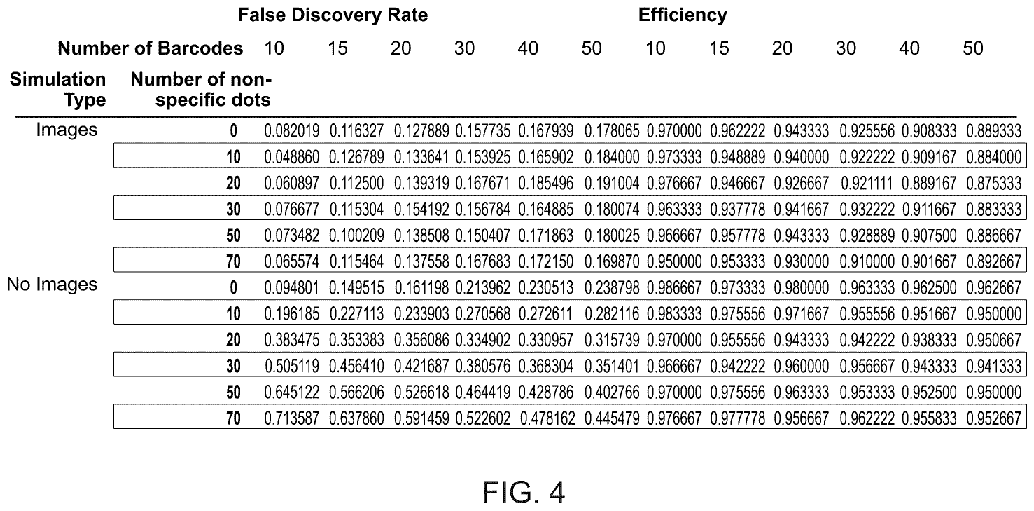

The simulations with Reed-Solomon codes showed promising results and suggested that it was possible to decode with much higher densities. Simulations 1-pixel areas of ISG experiments encoded with various Reed-Solomon codes and containing various numbers of barcodes show that the decodable density of ISG experiments can be dramatically increased by using Reed-Solomon codes with minimum distance of 4 or higher (FIG. 2, FIG. 7, FIG. 8, FIG. 9). A simulation that includes generated images over a larger 10×10 pixel grid where simulated transcripts appear at frequencies proportional to their FPKM values in bulk sequencing NIH3T3 cells indicates that these improvements will hold under more realistic conditions (FIG. 3, FIG. 4).

### Example 11

### Identifying Candidate Barcodes with Syndrome Decoding

To identify candidate barcodes, dynamic programming was used to find aligning dots across barcoding blocks that satisfy the error-correcting code's parity check equations (FIG. 1, FIG. 6). The algorithm is illustrated using a q-ary parity check code with four barcoding blocks and 20 pseudo-colors (Eng et al.). All possible combinations of pseudo-colors are allowed in the first three barcoding blocks, but the final pseudo-color is determined by the parity check equation:

$${c}_{4} = {c}_{1} + {c}_{2} - {c}_{3} ; \qquad Equation 8$$

where cᵢ denotes the pseudo-color of a barcode in barcoding block i, and addition and subtraction operations are modulo 20, the number of pseudo-colors. The parity check equation defines the syndrome,

$$s = {c}_{1} + {c}_{2} - {c}_{3} - {c}_{4} ; . \qquad Equation 9$$

such that it will be 0 if and only if the dots' pseudo-colors represent a codeword.

In general, linear error-correcting codes are defined by a parity check matrix, H, such that

$$H c = 0 ; \qquad Equation 10$$

Where C is the set of codewords in the code. The codewords, c, are vectors of symbols drawn from an alphabet of size q. Arithmetic operations are modulo-q. The parity check equation defines the syndrome,

$$s = H c ; \qquad Equation 11$$

Such that all elements of s will be 0 if and only if the dots' pseudo-colors represent a codeword. The linear nature of the syndrome computation allows all candidate barcodes to be found with dynamic programming, a technique that is also known by the more descriptive name, recursive optimization. This procedure involves breaking the matrix multiplication summing operations into steps and saving intermediate sums allowing us to reduce the number of arithmetic operations that must be performed.

The first step in decoding is to break up the identified dots into overlapping square tiles of width two times the search. The centers of each tile are spaced in a square grid with spacing of one search radius. This ensures that all collections of dots that are all located within a search radius of each other will be found in the search. To find candidate barcodes in each tile, a dynamic programming algorithm is used to sum syndrome components for all combinations of dots from different barcoding blocks that align to within the search radius.

The following example illustrates the simpler case of the dynamic programming algorithm where barcodes are probed in every barcoding block (FIG. 1, FIG. 6). First a one-element array is allocated for every dot in the first barcoding round containing that dot's contribution to a syndrome. For each dot, d, in subsequent i^{th} barcoding blocks (i>1), partial syndrome sum arrays were constructed for d by concatenating the partial syndrome sum arrays of dots found to be within one search radius of d's coordinates in barcoding block i−1 by a KDTree ball search, then adding the pseudo-color of d element-wise. Syndromes with value 0 in the final barcoding block represent candidate barcodes and the dots that contributed to it could be traced. Candidate barcodes were removed from consideration that either have any two dots farther apart than the search radius or are duplicates of a candidate barcode found in multiple tiles.

The following example illustrates the procedure for the more general and complex case where barcodes are not probed in every barcoding block (FIG. 1, FIG. 6). In this case, dots from each barcoding block must be aligned with dots from multiple previous barcoding blocks and track the number of dots contributing to each partial sum. To do this, partial sums for each dot are stored in one of w arrays where the elements in the i^{th} of the w arrays hold partial sums of pseudo-colors from i dots. The first array of partial sums for each dot contains just that dot's contribution to a syndrome. The i^{th} partial sums array for each dot, where i>1, is found by concatenating the (i−1)^{th} nested syndrome partial sum array for dots in previous barcoding blocks within the search radius, then adding the syndrome contribution of the searching dot element-wise. The w^{th} array holds the syndromes for paths of aligning dots. Entries in the w^{th} array with value 0 represent candidate barcodes and the dots that contributed to it can be traced. When allowing incomplete barcodes with 1 missing dot, all paths of length w−1 are traced and searched in a BKTree of codewords to check whether they are Hamming distance 1 from a valid codeword. Duplicate candidate barcodes found in multiple of the overlapping tiles and candidate barcodes are removed that have any two dots farther apart than the search radius.

### Example 12

### Syndrome Decoding for ISG Experiments Encoded by Linear Block Codes

The first step in decoding was to run Density-Based Spatial Clustering of Applications with Noise (DBSCAN) to find dense networks of dots across all hybridizations. To find candidate barcodes in each DBSCAN cluster, a dynamic programming algorithm was used to sum syndrome components for all combinations of dots from different barcoding blocks that align (FIG. 1, FIG. 6).

A Directed Acyclic Graph (DAG) was created, where each dot is a node and directed edges point from dots in higher numbered barcoding blocks to dots in lower numbered barcoding blocks. The DAG was implemented with KDTrees for each barcoding block containing the coordinates of dots that are in barcoding blocks of lesser index. The dots to which each dot has outgoing edges pointing to were enumerated by a KDTree ball search of fixed radius. The paths whose dots' barcoding block and pseudo-color values satisfied the parity check equations were perfect codepaths. When a dot from a barcode failed to readout or align, the result may have been imperfect codepaths that were missing one or two dots, but were still unambiguously closest to the correct codeword that they represent.

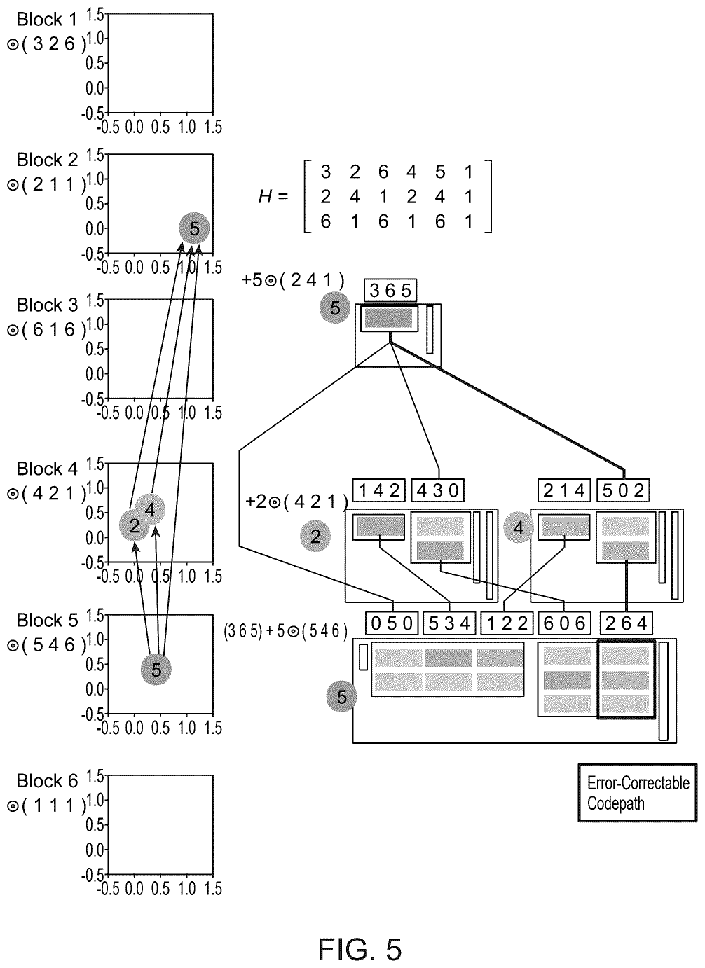

The maximum number of barcoding blocks over which an edge may point to avoid unnecessary computations that would not identify any additional codepaths were restricted. The highest indexed barcoding block of a dot in a decodable collection of dots could be any index w−t+1 or greater. The DAG included directed edges from dots in each barcoding block to dots that spatially align with them in indexed barcoding blocks up to n−w+t+1 less than itself (FIG. 5, FIG. 6).

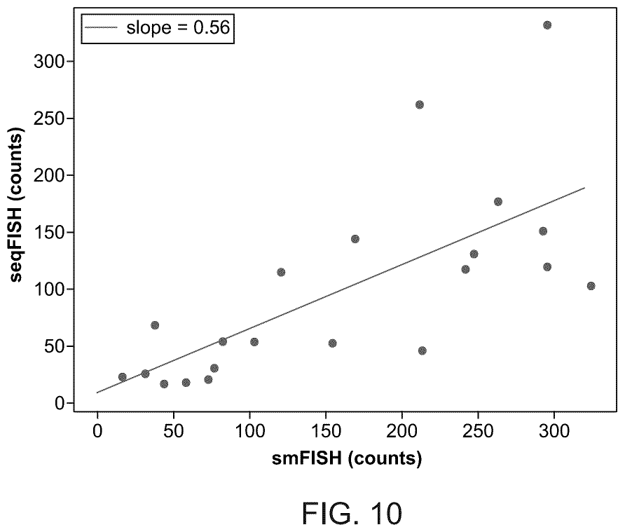

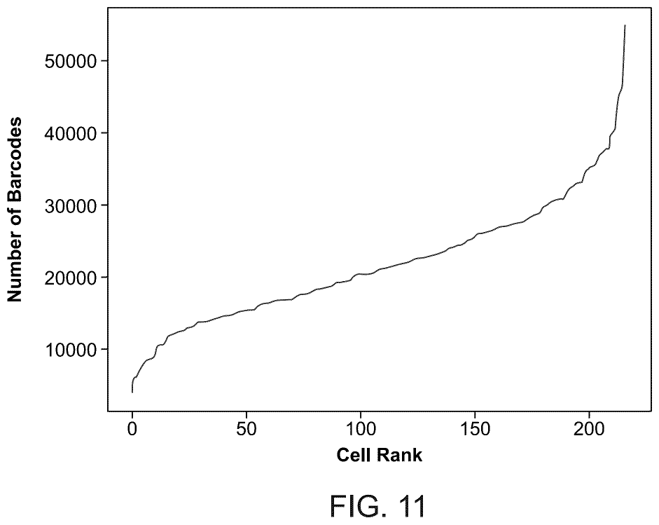

Dynamic programming was used to efficiently identify codepaths in the DAG. The parity check equations were recursively summed along every path of the DAG while saving intermediate sums to avoid making the same computation twice (FIG. 5, FIG. 6). The intermediate sums for each dot were stored in w separate arrays where the i^{th} array of intermediate sums contains the sum of syndrome components from i dots (FIG. 10).

For dots from each barcoding block starting with the first, the first intermediate sum array containing one element was allocated, the syndrome component of that dot. Each dot's i^{th} intermediate syndrome sum array from 2nd to w^{th} is found by adding its syndrome component element-wise to the concatenation of the (i−1)^{th} intermediate sum arrays of each dot to which it has an outgoing edge. Syndromes with value 0 in the final barcoding block indicate a perfect codepath, and the dots that contributed to it were traced. If decoding with drops is allowed, the method traced dots contributing to syndromes in the intermediate sum arrays of index w−t to index w−1 and used a standard decoding algorithm, or BKTree, to check in the message indicated by those dots whether it is correctable to a codeword (FIG. 5, FIG. 6). If so, then those dots constitute an imperfect codepath and were added as candidate barcodes. Candidate barcodes were removed from consideration that have any two dots farther apart than the search radius. After evaluating the sums for a dot, the w^{th} sum array of full syndromes was discarded.

To make efficient use of memory when performing the dynamic programming calculation of syndromes, it was important to minimize the number of intermediate sums that are allocated at any one time. Before starting the calculation, first, the number of dots that may be the terminal dot in a codepath for every dot in the DAG were counted. For every potentially terminal dot, the number of other potentially terminal dots that are within a search radius of it were also counted. An iteration was made through the potential terminal dots and the one with the fewest number of neighboring potentially terminal dots that have not had dots in their paths evaluated were chosen. The next step was to recursively evaluate intermediate sums for that dot and all dots reachable from it in the DAG that have not already had their intermediate sums evaluated. After evaluating the paths and identifying any codepaths, an update was made with the number of uncomputed potentially terminal dots in range of dot in lower numbered barcoding blocks within search radius of the dot. Any dots that has reached a count of zero uncomputed potentially terminal dots in its search radius have their intermediate sums deallocated.

In dense experiments, syndrome decoding will yield conflicting barcode candidates that include at least one of the same dots (FIG. 1). The densest regions may have large networks of conflicting barcode candidates. To find the non-conflicting barcodes that best explain the observed dots (FIG. 1), a cost function for each feasible solution was defined, wherein the cost function according to:

$$C = {\sum }_{b}^{B} ( {a}_{1} ( var ( {x}_{b} ) + var ( {y}_{b} ) ) + {a}_{2} var ( log ( {I}_{b} ) ) + {a}_{3} {δ}_{imperfect} ( b ) ) + {N}_{nd} . ; \qquad Equation 12$$

where B is the set of all barcode candidates in the network, b is a barcode candidate in B, and var (x_{b}), var (y_{b}), and var (log(I_{b})) are the variances of x-coordinates, y-coordinates and log intensities of each dot in the candidate barcode b. δ_{imperfect} is an indicator variable that is equal to 1 when b is an imperfect barcode and equal to 0 when it is a perfect barcode. a₁ is a position variance penalty; a₂ is a weight variance penalty; a₃ is a penalty for imperfect barcodes, are user set parameters. N_{nd} is a number of dots in the network that are not in a barcode candidate chosen by the solution. In some cases, it may be advantageous to add more terms to the cost function, for example a term scoring similarity in shape of the dots in each barcode candidate. An integer programming solver (Dunning, Huchette, and Lubin) was used to find the lowest cost set of barcode candidates that do not include the same readout dots.

Data was decoded from Eng et al. (FIG. 1), and the search radius was set to 4 pixels. Search radius can also be set for identifying barcode candidates as

$$r = \vert 3 \sqrt{\frac{4}{{a}_{1}}} . ; \qquad Equation 13$$

Networks were discarded that have more than 2500 candidate barcodes and have a ratio of candidate barcodes to bounding box area greater than 10. A range of reasonable values was tested for the cost function parameters to gauge the trade-off between efficiency and false positive rate for each combination of parameters.

### Example 13

### Simulation

Simulated data was generated by first drawing true locations for each target object from a uniform distribution on a square grid of pixels. The size of the grid differed in different simulations. For the simulation of seqFISH+ (Eng et al.) error correcting code every readout was successfully detected, but for the simulations of Reed-Solomon codes every readout had only a 0.95 probability of successful detection. The x and y coordinates of the dots successfully readout from each barcode were drawn from a normal distribution of standard deviation 0.5 pixels around the simulated true location of the barcode. The locations of nonspecific dots were drawn from a uniform distribution on the simulation area. To investigate the effects of optical density on decoding, some simulations attempted to decode the simulated dot locations directly and others rendered the dots as images, attempt to detect the dots in the images using a modified version of ADCG (Boyd et al.), then decode the dot locations recovered from the images. Dots are rendered in simulated images as Gaussian point spread functions with equal intensity and a sigma of 1.2 pixels. To evaluate the decoding performance in simulations, the efficiency and FDR were calculated as:

$$Efficiency = \frac{{\sum }_{i = 1}^{N} {x}_{i} - max ( {x}_{i} - {\hat{x}}_{i} , 0 )}{{\sum }_{i = 1}^{N} {x}_{i}} . ; \qquad Equation 14$$

$$\vert FDR = \frac{{\sum }_{i = 1}^{N} max ( {\hat{x}}_{i} - {x}_{i} , 0 )}{{\sum }_{i = 1}^{N} {\hat{x}}_{i}} . ; \qquad Equation 15$$

where xᵢ is the number of simulated transcript counts and \hat{x}ᵢ is the number decoded transcript counts for the i^{th} type of object in the simulation, and N is the number of types of objects probed in the simulations. 70 replicates of simulated data encoded using the error-correcting code as in (Eng et al.) in a 5×5 pixel ROI with various densities of barcodes and non-specific dots with locations drawn from the uniform distribution (FIG. 3).

### Example 14

### Estimating False Discovery Rate (FDR) in Real Data

To measure the FDR, codewords were used in the experiment's codebook that do not encode a gene and are not probed for. These unassigned codewords served as negative controls. When the images were decoded, a search was made for barcodes encoding negative control codewords in addition to gene encoding codewords in the codebook. To estimate how many of the gene encoding barcodes were false discoveries, an assumption was made that barcodes encoding any codeword were equally likely to be found in error. To estimate the FDR, the equation:

$$\hat{F} = \frac{{M}_{g} ( \frac{{N}_{nc}}{{M}_{nc}} )}{{N}_{g}} ; \qquad Equation 16$$

was used, where N_{g} and N_{nc} are the number of gene encoding and negative control barcodes found, and M_{g} and Mₙ are the number of gene encoding and negative control codewords in the codebook. An assumption was made that the mean number of false counts per codeword is the same for all codewords, allowing an estimation of the number of false positive counts by multiplying the mean number of false counts per negative control codeword N_{nc}/M_{nc}, by M_{g}. To estimate the proportion of gene encoding barcodes that are found in error, the expected number of false counts was divided by the total number of observed positive counts.

### Example 15

### Fitting Barcodes to Images Stacks

The first step in the process was to do a grid search to determine which coordinates in each image are surrounded by pixels bright enough that the coordinates may conceivably be the coordinates of a dot. This was implemented by convolving the shape of a Gaussian point spread function with the image, and selecting the grid coordinates producing values in the convolution above a threshold.

Second, the candidate locations for dots in each image were thinned using lasso regression where the data vector is the pixel values of the image, and the explanatory variables are sparse vectors of the normalized value of the point spread function at each candidate location. Grid locations whose point spread functions were assigned non-zero values in the model with the least stringent regulation parameter were removed from the candidates list.

Next, candidate barcodes were identified by syndrome decoding with a non-zero search radius. The candidate barcodes were thinned by running a LASSO regression where the entries in the observation vector are the observed pixel values in the entire image stack. Sparse explanatory vectors for each candidate barcode held normalized values of the PSFs that comprise the candidate barcode. Again, candidate barcodes whose coefficients in the model were zero at the least stringent value of the regulation parameter were discarded.

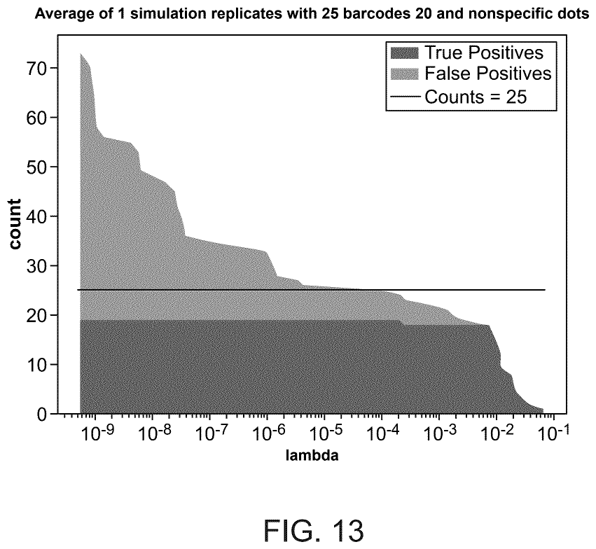

Finally, a L0-regulated regression model was run to choose the final set of barcodes from the candidates list (FIG. 13). The value of the regulation parameter (λ) controls the sparsity of the solution. We find that λ˜0.01 results in a good fit that correctly identifies most barcodes without overfitting and erroneously decoding false barcodes.

### Example 16

### Simulations and Limits on Resolving Dense Barcodes from Image Fitting

The ADCG fitting algorithm used cannot resolve separate point spread functions in the same optical image located within a full width at half max of each other. Such overlapping dots in the same image may also cause fitting errors resulting in dots whose fit coordinates do not correspond with those of the actual molecules. To investigate how much errors in image fitting impact decoding, synthetic image data was simulated and dot location data modeled off of the previously published 10,000 gene seqFISH+ experiment (FIG. 12) (Eng et al.). In these simulations, not only were simulated dot locations fed directly into the decoder, but synthetic images were produced with Gaussian point spread functions drawn at the simulated dot locations and the images were fit with ADCG, then the fit dot locations recovered from the image fitting were decoded. 70 replicates of seqFISH+ data were simulated in a 5×5 pixel ROI with various densities of barcodes and non-specific dots with locations drawn from the uniform distribution. The x and y coordinates of dots readout from each barcode are drawn from a normal distribution of standard deviation 0.5 pixels around the simulated true location of the barcode. Simulated images are generated by drawing both nonspecific and barcode dots as Gaussian point spread functions with a sigma of 1.2 pixels and the same brightness. ADCG attempts to recover the dot locations from the simulated images, and both the ADCG recovered dots and the directly simulated dot locations are decoded by syndrome decoding with a position variance penalty of 6 and a log brightness variance penalty of 0. A decrease in decoding efficiency was found and a decrease in sensitivity to extra non-specific dots in the image-based simulations compared to the image-less simulations (FIG. 12). Performance metrics for the simulation were calculated as described in the methods section.

### REFERENCES

The following references are incorporated by reference in their entirety.
Andersson, Axel, Ferran Diego, Fred A. Hamprecht, and Carolina Wählby. 2021. “ISTDECO: In Situ Transcriptomics Decoding by Deconvolution.” Bioinformatics. http://biorxiv.org/lookup/doi/10.1101/2021.03.01.433040.
Boyd, Nicholas, Geoffrey Schiebinger, and Benjamin Recht. 01/2017. “The Alternating Descent Conditional Gradient Method for Sparse Inverse Problems.” SIAM Journal on Optimization: A Publication of the Society for Industrial and Applied Mathematics 27 (2): 616-39.
John P Bryan, Loïc Binan, Cai McCann, Yonina C Eldar, Samouil L Farhi, Brian Cleary, Optimization-based decoding of Imaging Spatial Transcriptomics data, Bioinformatics, Volume 39, Issue 6, June 2023, btad362, https://doi.org/10.1093/bioinformatics/btad362
Chen, Kok Hao, Alistair N. Boettiger, Jeffrey R. Moffitt, Siyuan Wang, and Xiaowei Zhuang. 2015. “Spatially Resolved, Highly Multiplexed RNA Profiling in Single Cells.” Science 348 (6233): aaa6090.
Chen, Shuonan, Jackson Loper, Xiaoyin Chen, Alex Vaughan, Anthony M. Zador, and Liam Paninski. 2021. “BARcode DEmixing through Non-Negative Spatial Regression (BarDensr).” Edited by Emma Claire Robinson. PLOS Computational Biology 17 (3): e1008256.
Dunning, Iain, Joey Huchette, and Miles Lubin. 01/2017. “JUMP: A Modeling Language for Mathematical Optimization.” SIAM Review 59 (2): 295-320.
Eng, Chee-Huat Linus, Michael Lawson, Qian Zhu, Ruben Dries, Noushin Koulena, Yodai Takei, Jina Yun, et al. 4/2019. “Transcriptome-Scale Super-Resolved Imaging in Tissues by RNA seqFISH+.” Nature 568 (7751): 235-39.
Eng, Chee-Huat Linus, Sheel Shah, Julian Thomassie, and Long Cai. 12/2017. “Profiling the Transcriptome with RNA SPOTs.” Nature Methods 14 (12): 1153-55.
Femino, Andrea M., Fredric S. Fay, Kevin Fogarty, and Robert H. Singer. 1998. “Visualization of Single RNA Transcripts in Situ” 280:7.
Gataric, Milana, Jun Sung Park, Tong Li, Vasyl Vaskivskyi, Jessica Svedlund, Carina Strell, Kenny Roberts, et al. 2021. “POSTcode: Probabilistic Image-Based Spatial Transcriptomics Decoder.” Bioinformatics. Available at: biorxiv.org/lookup/doi/10.1101/2021.10.12.464086.
Groth, E. J. 5/1986. “A Pattern-Matching Algorithm for Two-Dimensional Coordinate Lists.” The Astronomical Journal 91:1244.
Guizar-Sicairos, Manuel, Samuel T. Thurman, and James R. Fienup. 2008. “Efficient Subpixel Image Registration Algorithms.” Optics Letters. Available at: doi.org/10.1364/ol.33.000156.
Lubeck, Eric, Ahmet F. Coskun, Timur Zhiyentayev, Mubhij Ahmad, and Long Cai. 4/2014. “Single-Cell in Situ RNA Profiling by Sequential Hybridization.” Nature Methods 11 (4): 360-61.
Moffitt, Jeffrey R., Junjie Hao, Guiping Wang, Kok Hao Chen, Hazen P. Babcock, and Xiaowei Zhuang. 2016. “High-Throughput Single-Cell Gene-Expression Profiling with Multiplexed Error-Robust Fluorescence in Situ Hybridization.” Proceedings of the National Academy of Sciences of the United States of America 113 (39): 11046-51.
Moffitt JR, Hao J, Bambah-Mukku D, Lu T, Dulac C, Zhuang X. High-performance multiplexed fluorescence in situ hybridization in culture and tissue with matrix imprinting and clearing. Proc Natl Acad Sci USA. 2016 Dec. 13;113(50):14456-14461. doi: 10.1073/pnas.1617699113. Epub 2016 Nov. 22. PMID: 27911841; PMCID: PMC5167177.
Mölder, Felix, Kim Philipp Jablonski, Brice Letcher, Michael B. Hall, Christopher H. Tomkins-Tinch, Vanessa Sochat, Jan Forster, et al. 2021. “Sustainable Data Analysis with Snakemake.” F1000Research 10 (January): 33.
Raj, Arjun, Patrick van den Bogaard, Scott A. Rifkin, Alexander van Oudenaarden, and Sanjay Tyagi. 10/2008. “Imaging Individual mRNA Molecules Using Multiple Singly Labeled Probes.” Nature Methods 5 (10): 877-79.
Sage, Daniel, Thanh-An Pham, Hazen Babcock, Tomas Lukes, Thomas Pengo, Jerry Chao, Ramraj Velmurugan, et al. 5/2019. “Super-Resolution Fight Club: Assessment of 2D and 3D Single-Molecule Localization Microscopy Software.” Nature Methods 16 (5): 387-95.
Shah, Sheel, Yodai Takei, Wen Zhou, Eric Lubeck, Jina Yun, Chee-Huat Linus Eng, Noushin Koulena, et al. 07/2018. “Dynamics and Spatial Genomics of the Nascent Transcriptome by Intron seqFISH.” Cell 174 (2): 363-76.e16.
Sternberg. 1983. “Biomedical Image Processing.” Computer 16 (1): 22-34.
Takei, Yodai, Jina Yun, Shiwei Zheng, Noah Ollikainen, Nico Pierson, Jonathan White, Sheel Shah, et al. 2021. “Integrated Spatial Genomics Reveals Global Architecture of Single Nuclei.” Nature 590 (7845): 344-50.
Takei, Yodai, Shiwei Zheng, Jina Yun, Sheel Shah, Nico Pierson, Jonathan White, Simone Schindler, Carsten H. Tischbirek, Guo-Cheng Yuan, and Long Cai. 2021. “Single-Cell Nuclear Architecture across Cell Types in the Mouse Brain.” Science 374 (6567): 586-94.
Walt, Stéfan van der, Johannes L. Schönberger, Juan Nunez-Iglesias, François Boulogne, Joshua D. Warner, Neil Yager, Emmanuelle Gouillart, and Tony Yu. 2014. “Scikit-Image: Image Processing in Python.” PeerJ 2 (June): e453.
T. Chang, W. Han, M. Jiang, J. Li, Z. Liao, M. Tang, J. Zhang, J. Shen, Z. Chen, P. Fei, X. Ren, Y. Pang, G. Wang, J. Wang, Y. Huang, Rapid and signal crowdedness-robust in situ sequencing through hybrid block coding, Proc. Natl. Acad. Sci. U.S.A.
Daniel Gyllborg, Christoffer Mattsson Langseth, Xiaoyan Qian, Eunkyoung Choi, Sergio Marco Salas, Markus M Hilscher, Ed S Lein, Mats Nilsson, Hybridization-based in situ sequencing (HybISS) for spatially resolved transcriptomics in human and mouse brain tissue, Nucleic Acids Research, Volume 48, Issue 19, 4 Nov. 2020, Page e112, Available at: doi.org/10.1093/nar/gkaa792
Xiao Wang et al., Three-dimensional intact-tissue sequencing of single-cell transcriptional states. Science361,eaat5691 (2018).DOI:10.1126/science.aat5691 120 (47) e2309227120,
C. Xia, J. Fan, G. Emanuel, J. Hao, X. Zhuang, Spatial transcriptome profiling by MERFISH reveals subcellular RNA compartmentalization and cell cycle-dependent gene expression, Proc. Natl. Acad. Sci. U.S.A. 116 (39) 19490-19499, Available at: doi.org/10.1073/pnas. 1912459116 (2019).

Error Control Coding Second Edition. Shu Lin, Daniel J. Costello Jr. Pearson 2005

The Theory of Error-Correcting Codes. F.J. MacWilliams, N.J.A. Sloane. North Holland Publishing Company 1977. New York, New York.

1. A computer-implemented method to assign barcodes from images of dots representing fluorescent probes interacting with molecular targets, to a plurality of molecular targets, the method comprising:
(i) using a computer, tracing dots between images within a search radius (r) and assigning dots to barcodes to form a plurality of candidate barcodes;
(ii) using a computer, assigning a cost to the plurality of candidate barcodes to obtain an assigned cost;
(iii) using a computer, for the plurality of candidate barcodes, assigning a penalty to unused dots in each of the images to obtain a penalty cost;
(iv) using a computer, choosing a trial solution, the trial solution comprising a set of candidate barcodes with the smallest cost; and
(v) using a computer, assigning the candidate barcodes, selected by the trial solution, with the smallest cost to the molecular targets.

2. The method of claim 1, further comprising for each candidate barcode in the plurality of candidate barcodes, adding the assigned cost to the penalty cost.

3. The method of claim 1, further comprising summing the assigned cost and the penalty cost for each of the plurality of candidate barcodes to obtain a respective total cost.

4. The method of claim 1, wherein the dots in each of the images are fitted to a function that determines their spatial coordinates.

5. The method of claim 4, wherein the function is Gaussian or Airy.

6. The method of claim 1, further comprising aligning each of the images using one or more reference positions in each image.

7. The method of claim 1, wherein candidate barcodes are selected from linear codes.

8. The method of claim 7, wherein the linear codes are selected from the group consisting of BCH codes and Hamming codes.

9. The method of claim 7, wherein the linear code is a Reed-Solomon code.

10. The method of claim 1, further comprising assigning each dot a pseudo-color value or symbol in each image.

11. The method of claim 10, wherein the symbol is an element of a codeword, wherein the codeword identifies the molecular target.

12. The method of claim 1, comprising identifying each candidate barcode using a coding scheme that does not have parity checks.

13. The method of claim 1, further comprising resolving conflicting candidate barcodes that use at least one of the same dots.

14. The method of claim 1, further comprising identifying candidate barcodes by summing syndromes.

15. The method of claim 1, wherein the cost is an error function measuring the deviation of the observed pixel intensities from the pixel intensities predicted by trial solutions and is determined according to:
$$C = f ( {N}_{nd} )$$
wherein N_{nd} is a number of dots in the images that are not used in the candidate barcodes, and f is a function.

16. The method of claim 1, wherein the cost is determined according to:
$$C = {\sum }_{b}^{B} ( {a}_{1} ( var ( {x}_{b} ) + var ( {y}_{b} ) ) + {a}_{2} var ( log ( {I}_{b} ) ) + {a}_{3} {δ}_{imperfect} ( b ) ) + {N}_{nd} .$$
where B is the set of all barcode candidates in the trial solution, b is a barcode candidate in B, and var (x_{b}), var (y_{b}), and var (log(I_{b})) are the variances of x-coordinates; y-coordinates and log intensities of each dot in the candidate barcode b; δ_{imperfect} is an indicator variable that is equal to 1 when b is an imperfect barcode and equal to 0 when it is a perfect barcode; a₁ is a position variance penalty; a₂ is a weight variance penalty; a₃ is a penalty for imperfect barcodes, are user set parameters; N_{nd} is a number of dots in the images that are not used in the candidate barcodes.

17. The method of claim 13, further comprising using dynamic programming to identify codepaths, wherein the codepaths are candidate barcodes.

18. The method of claim 1, wherein the cost is determined using pixel intensities in the images and deviations from the pixel intensities predicted by the trial solutions.

19. The method of claim 1, wherein the cost is determined using regularized regression to select candidate barcodes that account for dots in images.

20. The method of claim 1, where spatial positions of the candidate barcodes can be determined by taking mean, median, or other functions, of the spatial positions of the dots that make up the candidate barcodes.
